SEQUENCE LISTING (193 sequences)

SEQ ID NO: 1 - protein, 202 aa, Artificial Sequence
  Description of Artificial Sequence Synthetic polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTGHHHHHH

SEQ ID NO: 2 - protein, 248 aa, Mus sp.
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGS

SEQ ID NO: 3 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 193 aa, Mus sp.
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 313 aa, Mus sp.
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - protein, 449 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 8 - protein, 585 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 9 - protein, 459 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 10 - protein, 579 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 11 - protein, 786 aa, Artificial Sequence
  Description of Artificial Sequence Synthetic polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 790 aa, Artificial Sequence
  Description of Artificial Sequence Synthetic polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 13 - protein, 10 aa, Unknown
  Description of Unknown MMP-response sequence
GLLSGRSDNH

SEQ ID NO: 14 - protein, 6 aa, Unknown
  Description of Unknown thrombin-responsive sequence
LVPRGS

SEQ ID NO: 15 - protein, 469 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 16 - protein, 463 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 17 - protein, 451 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 18 - protein, 481 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 19 - protein, 639 aa, Homo sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 20 - protein, 719 aa, Mus sp.
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQISRLSIIFASLGSFASVLLVGSLGYIGLNRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 21 - protein, 839 aa, Homo sapiens
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 22 - protein, 851 aa, Mus sp.
NIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEEPVGILDIWYMKQDIDYDRQQISLFWKSLNPSEARGKILHYQVTLQEVTKKTTLQNTTRHTSWTRVIPRTGAWTASVSAANSKGASAPTHINIVDLCGTGLLAPHQVSAKSENMDNILVTWQPPKKADSAVREYIVEWRALQPGSITKFPPHWLRIPPDNMSALISENIKPYICYEIRVHALSESQGGCSSIRGDSKHKAPVSGPHITAITEKKERLFISWTHIPFPEQRGCILHYRIYWKERDSTAQPELCEIQYRRSQNSHPISSLQPRVTYVLWMTAVTAAGESPQGNEREFCPQGKANWKAFVISSICIAIITVGTFSIRYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 23 - protein, 134 aa, Homo sapiens
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 24 - protein, 148 aa, Mus sp.
PTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 25 - protein, 133 aa, Mus sp.
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 26 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 27 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 28 - protein, 214 aa, Mus sp.
AVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKE

SEQ ID NO: 29 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 30 - protein, 215 aa, Mus sp.
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 31 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 32 - protein, 241 aa, Mus sp.
WSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEA

SEQ ID NO: 33 - protein, 228 aa, Homo sapiens [sequence omitted]

SEQ ID NO: 34 - protein, 229 aa, Mus sp.
ALTSTEDPEPPSVPVPTNVLIKSYNLNPVVCWEYQNMSQTPIFTVQVKVYSGSWTDSCTNISDHCCNIYEQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKDS

SEQ ID NO: 35 - protein, 220 aa, Homo sapiens
SQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 36 - protein, 313 aa, Mus sp.
ASSPDSFSQLAAPLNPRLHLYNDEQILTWEPSPSSNDPRPVVYQVEYSFIDGSWHRLLEPNCTDITETKCDLTGGGRLKLFPHPFTVFLRVRAKRGNLTSKWVGLEPFQHYENVTVGPPKNISVTPGKGSLVIHFSPPFDVFHGATFQYLVHYWEKSETQQEQVEGPFKSNSIVLGNLKPYRVYCLQTEAQLILKNKKIRPHGLLSNVSCHETTANASARLQQVILIPLGIFALLLGLTGACFTLFLKYQSRVKYWFQAPPNIPEQIEEYLKDPDQFILEVLDKDGSPKEDSWDSVSIISSPEKERDDVLQTP

SEQ ID NO: 37 - protein, 196 aa, Unknown
  Description of Unknown vWF A3 sequence
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 38 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 39 - protein, 222 aa, Homo sapiens
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 40 - protein, 344 aa, Homo sapiens
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 41 - protein, 10 aa, Homo sapiens
LRELHLDNNC

SEQ ID NO: 42 - protein, 584 aa, Mus sp.
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 43 - protein, 608 aa, Homo sapiens
KWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 44 - protein, 10 aa, Bos sp.
LRELHLNNNC

SEQ ID NO: 45 - protein, 193 aa, Homo sapiens
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 46 - protein, 205 aa, Homo sapiens
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 47 - protein, 8 aa, Artificial Sequence
  Description of Artificial Sequence Synthetic peptide
GGGSGGGS

SEQ ID NO: 48 - protein, 32 aa, Artificial Sequence
  Description of Artificial Sequence Synthetic polypeptide
  MISC_FEATURE (1)..(32): This sequence may encompass 1-8 “Gly Gly Gly Ser” repeating units
GGGSGGGSGGGSGGGSGGGSGGGSGGGSGGGS

SEQ ID NO: 49 - protein, 8 aa, Unknown
  Description of Unknown tumor-associated protease site sequence
LSGRSDNH

SEQ ID NO: 50 - protein, 7 aa, Unknown
  Description of Unknown tumor-associated protease site sequence
VPLSLYS

SEQ ID NO: 51 - protein, 6 aa, Unknown
  Description of Unknown tumor-associated protease site sequence
PLGLAG

SEQ ID NO: 52 - protein, 10 aa, Unknown
  Description of Unknown tumor-associated protease site sequence
VLVPMAMMAS

SEQ ID NO: 53 - protein, 7 aa, Unknown
  Description of Unknown tumor-associated protease site sequence
  MOD_RES (1)..(2): Any amino acid
  MOD_RES (6)..(6): Ala or Val
  MOD_RES (7)..(7): Any amino acid
XXQARXX

SEQ ID NO: 54 - protein, 4 aa, Unknown
  Description of Unknown tumor-associated protease site sequence
AGPR

SEQ ID NO: 55 - protein, 4 aa, Unknown
  Description of Unknown tumor-associated protease site sequence
AANL

SEQ ID NO: 56 - protein, 4 aa, Unknown
  Description of Unknown tumor-associated protease site sequence
PTNL

SEQ ID NO: 57 - protein, 8 aa, Unknown
  Description of Unknown tumor-associated protease site sequence
TSGRSANP

SEQ ID NO: 58 - protein, 39 aa, Artificial Sequence
  Description of Artificial Sequence Synthetic polypeptide
GASTSVDELQAEVDQLQDENYALKTKVAQLRKKVEKLSE

SEQ ID NO: 59 - protein, 38 aa, Artificial Sequence
  Description of Artificial Sequence Synthetic polypeptide
GASTTVAQLRERVKTLRAQNYELESEVQRLREQVAQLA

SEQ ID NO: 60 - protein, 42 aa, Artificial Sequence
  Description of Artificial Sequence Synthetic polypeptide
EACGASTSVDELQAEVDQLQDENYALKTKVAQLRKKVEKLSE

SEQ ID NO: 61 - protein, 41 aa, Artificial Sequence
  Description of Artificial Sequence Synthetic polypeptide
EACGASTTVAQLRERVKTLRAQNYELESEVQRLREQVAQLA

SEQ ID NO: 62 - protein, 40 aa, Artificial Sequence
  Description of Artificial Sequence Synthetic polypeptide
LEIEAAFLERENTALETRVAELRQRVQRARNRVSQYRTRY

SEQ ID NO: 63 - protein, 40 aa, Artificial Sequence
  Description of Artificial Sequence Synthetic polypeptide
LEIRAAFLRQRNTALRTEVAELEQEVQRLENEVSQYETRY

SEQ ID NO: 64 - protein, 45 aa, Artificial Sequence
  Description of Artificial Sequence Synthetic polypeptide
EACGALEIEAAFLERENTALETRVAELRQRVQRARNRVSQYRTRY

SEQ ID NO: 65 - protein, 45 aa, Artificial Sequence
  Description of Artificial Sequence Synthetic polypeptide
EACGALEIRAAFLRQRNTALRTEVAELEQEVQRLENEVSQYETRY

SEQ ID NO: 66 - protein, 40 aa, Artificial Sequence
  Description of Artificial Sequence Synthetic polypeptide
LEIRAAFLRRRNTALRTRVAELRQRVQRLRNIVSQYETRY

SEQ ID NO: 67 - protein, 40 aa, Artificial Sequence
  Description of Artificial Sequence Synthetic polypeptide
LEIEAAFLEQENTALETEVAELEQEVQRLENIVSQYETRY

SEQ ID NO: 68 - protein, 45 aa, Artificial Sequence
  Description of Artificial Sequence Synthetic polypeptide
EACGALEIRAAFLRRRNTALRTRVAELRQRVQRLRNIVSQYETRY

SEQ ID NO: 69 - protein, 45 aa, Artificial Sequence
  Description of Artificial Sequence Synthetic polypeptide
EACGALEIEAAFLEQENTALETEVAELEQEVQRLENIVSQYETRY

SEQ ID NO: 70 - protein, 38 aa, Artificial Sequence
  Description of Artificial Sequence Synthetic polypeptide
QGASTSVDELQAEVDQLEDENYALKTKVAQLRKKVEKL

SEQ ID NO: 71 - protein, 38 aa, Artificial Sequence
  Description of Artificial Sequence Synthetic polypeptide
QGASTTVAQLEEKVKTLRAENYELKSEVQRLEEQVAQL

SEQ ID NO: 72 - protein, 40 aa, Artificial Sequence
  Description of Artificial Sequence Synthetic polypeptide
EACGASTSVDELQAEVDQLEDENYALKTKVAQLRKKVEKL

SEQ ID NO: 73 - protein, 40 aa, Artificial Sequence
  Description of Artificial Sequence Synthetic polypeptide
EACGASTTVAQLEEKVKTLRAENYELKSEVQRLEEQVAQL

SEQ ID NO: 74 - protein, 42 aa, Artificial Sequence
  Description of Artificial Sequence Synthetic polypeptide
AGLTDTLQAETDQLEDKKSALQTEIANLLKEKEKLEFILAAH

SEQ ID NO: 75 - protein, 42 aa, Artificial Sequence
  Description of Artificial Sequence Synthetic polypeptide
AGRIARLEEKVKTLKAQNSELASTANMLREQVAQLKQKVMNY

SEQ ID NO: 76 - protein, 46 aa, Artificial Sequence
  Description of Artificial Sequence Synthetic polypeptide
EACGAGLTDTLQAETDQLEDKKSALQTEIANLLKEKEKLEFILAAH

SEQ ID NO: 77 - protein, 46 aa, Artificial Sequence
  Description of Artificial Sequence Synthetic polypeptide
EACGAGRIARLEEKVKTLKAQNSELASTANMLREQVAQLKQKVMNY

SEQ ID NO: 78 - protein, 29 aa, Artificial Sequence
  Description of Artificial Sequence Synthetic peptide
GKIAALKQKIAALKYKNAALKKKIAALKQ

SEQ ID NO: 79 - protein, 29 aa, Artificial Sequence
  Description of Artificial Sequence Synthetic peptide
GEIAALEQEIAALEKENAALEWEIAALEQ

SEQ ID NO: 80 - protein, 34 aa, Artificial Sequence
  Description of Artificial Sequence Synthetic polypeptide
EACGAGKIAALKQKIAALKYKNAALKKKIAALKQ

SEQ ID NO: 81 - protein, 34 aa, Artificial Sequence
  Description of Artificial Sequence Synthetic polypeptide
EACGAGEIAALEQEIAALEKENAALEWEIAALEQ

SEQ ID NO: 82 - protein, 15 aa, Artificial Sequence
  Description of Artificial Sequence Synthetic peptide
GLNDIFEAQKIEWHE

SEQ ID NO: 83 - protein, 26 aa, Artificial Sequence
  Description of Artificial Sequence Synthetic peptide
KRRWKKNFIAVSAANRFKKISSSGAL

SEQ ID NO: 84 - protein, 6 aa, Artificial Sequence
  Description of Artificial Sequence Synthetic peptide
EEEEEE

SEQ ID NO: 85 - protein, 8 aa, Artificial Sequence
  Description of Artificial Sequence Synthetic peptide
DYKDDDDK

SEQ ID NO: 86 - protein, 9 aa, Artificial Sequence
  Description of Artificial Sequence Synthetic peptide
YPYDVPDYA

SEQ ID NO: 87 - protein, 6 aa, Artificial Sequence
  Description of Artificial Sequence Synthetic 6xHis tag
HHHHHH

SEQ ID NO: 88 - protein, 10 aa, Artificial Sequence
  Description of Artificial Sequence Synthetic peptide
EQKLISEEDL

SEQ ID NO: 89 - protein, 18 aa, Artificial Sequence
  Description of Artificial Sequence Synthetic peptide
TKENPRSNQEESYDDNES

SEQ ID NO: 90 - protein, 15 aa, Artificial Sequence
  Description of Artificial Sequence Synthetic peptide
KETAAAKFERQHMDS

SEQ ID NO: 91 - protein, 38 aa, Artificial Sequence
  Description of Artificial Sequence Synthetic polypeptide
MDEKTTGWRGGHVVEGLAGELEQLRARLEHHPQGQREP

SEQ ID NO: 92 - protein, 13 aa, Artificial Sequence
  Description of Artificial Sequence Synthetic peptide
SLAELLNAGLGGS

SEQ ID NO: 93 - protein, 8 aa, Artificial Sequence
  Description of Artificial Sequence Synthetic peptide
TQDPSRVG

SEQ ID NO: 94 - protein, 8 aa, Artificial Sequence
  Description of Artificial Sequence Synthetic peptide
WSHPQFEK

SEQ ID NO: 95 - protein, 6 aa, Artificial Sequence
  Description of Artificial Sequence Synthetic peptide
CCPGCC

SEQ ID NO: 96 - protein, 14 aa, Artificial Sequence
  Description of Artificial Sequence Synthetic peptide
GKPIPNPLLGLDST

SEQ ID NO: 97 - protein, 11 aa, Artificial Sequence
  Description of Artificial Sequence Synthetic peptide
YTDIEMNRLGK

SEQ ID NO: 98 - protein, 8 aa, Artificial Sequence
  Description of Artificial Sequence Synthetic peptide
DLYDDDDK

SEQ ID NO: 99 - protein, 16 aa, Artificial Sequence
  Description of Artificial Sequence Synthetic peptide
TDKDMTITFTNKKDAE

SEQ ID NO: 100 - protein, 13 aa, Artificial Sequence
  Description of Artificial Sequence Synthetic peptide
AHIVMVDAYKPTK

SEQ ID NO: 101 - protein, 12 aa, Artificial Sequence
  Description of Artificial Sequence Synthetic peptide
KLGDIEFIKVNK

SEQ ID NO: 102 - protein, 13 aa, Artificial Sequence
  Description of Artificial Sequence Synthetic peptide
GAPVPYPDPLEPR

SEQ ID NO: 103 - protein, 455 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 104 - protein, 459 aa, Artificial Sequence; identical to SEQ ID NO: 9
  Description of Artificial Sequence Synthetic polypeptide

SEQ ID NO: 105 - protein, 475 aa, Artificial Sequence
  Description of Artificial Sequence Synthetic polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 106 - protein, 469 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 107 - protein, 457 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 108 - protein, 487 aa, Artificial Sequence
  Description of Artificial Sequence Synthetic polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 109 - protein, 8 aa, Unknown
  Description of Unknown tumor-associated protease site sequence
YLGRSYKV

SEQ ID NO: 110 - protein, 8 aa, Unknown
  Description of Unknown tumor-associated protease site sequence
TSQVNGLN

SEQ ID NO: 111 - protein, 8 aa, Unknown
  Description of Unknown tumor-associated protease site sequence
APPPVVLL

SEQ ID NO: 112 - protein, 5 aa, Unknown
  Description of Unknown tumor-associated protease site sequence
GGGGF

SEQ ID NO: 113 - protein, 8 aa, Unknown
  Description of Unknown tumor-associated protease site sequence
TPEHVVPY

SEQ ID NO: 114 - protein, 9 aa, Unknown
  Description of Unknown tumor-associated protease site sequence
PRSFFRLGK

SEQ ID NO: 115 - protein, 8 aa, Unknown
  Description of Unknown tumor-associated protease site sequence
PQGRIVGG

SEQ ID NO: 116 - protein, 8 aa, Unknown
  Description of Unknown tumor-associated protease site sequence
EVLLSWAV

SEQ ID NO: 117 - protein, 8 aa, Unknown
  Description of Unknown tumor-associated protease site sequence
PVSLSYRC

SEQ ID NO: 118 - protein, 8 aa, Unknown
  Description of Unknown tumor-associated protease site sequence
LRELHLDN

SEQ ID NO: 119 - protein, 4 aa, Unknown
  Description of Unknown tumor-associated protease site sequence
AAPV

SEQ ID NO: 120 - protein, 8 aa, Unknown
  Description of Unknown tumor-associated protease site sequence
IPENFFGV

SEQ ID NO: 121 - protein, 8 aa, Unknown
  Description of Unknown tumor-associated protease site sequence
PRFKIIGG

SEQ ID NO: 122 - protein, 8 aa, Unknown
  Description of Unknown tumor-associated protease site sequence
MLEDEASG

SEQ ID NO: 123 - protein, 8 aa, Unknown
  Description of Unknown tumor-associated protease site sequence
KQSRKFVP

SEQ ID NO: 124 - protein, 8 aa, Unknown
  Description of Unknown tumor-associated protease site sequence
RQARVVGG

SEQ ID NO: 125 - protein, 7 aa, Unknown
  Description of Unknown tumor-associated protease site sequence
GKAFRRL

SEQ ID NO: 126 - protein, 8 aa, Unknown
  Description of Unknown tumor-associated protease site sequence
GPLGLWAQ

SEQ ID NO: 127 - protein, 8 aa, Unknown
  Description of Unknown tumor-associated protease site sequence
SLGRKIQI

SEQ ID NO: 128 - protein, 5 aa, Unknown
  Description of Unknown tumor-associated protease site sequence
PVSLR

SEQ ID NO: 129 - protein, 8 aa, Unknown
  Description of Unknown tumor-associated protease site sequence
AAATSIAM

SEQ ID NO: 130 - protein, 8 aa, Unknown
  Description of Unknown tumor-associated protease site sequence
AAGAMFLE

SEQ ID NO: 131 - protein, 8 aa, Unknown
  Description of Unknown tumor-associated protease site sequence
EAAAATSI

SEQ ID NO: 132 - protein, 5 aa, Unknown
  Description of Unknown tumor-associated protease site sequence
PRHLR

SEQ ID NO: 133 - protein, 6 aa, Unknown
  Description of Unknown tumor-associated protease site sequence
PRGLRK

SEQ ID NO: 134 - protein, 8 aa, Unknown
  Description of Unknown tumor-associated protease site sequence
VPLSLYSG

SEQ ID NO: 135 - protein, 6 aa, Unknown
  Description of Unknown tumor-associated protease site sequence
PRGLRP

SEQ ID NO: 136 - protein, 6 aa, Unknown
  Description of Unknown tumor-associated protease site sequence
PRHLRN

SEQ ID NO: 137 - protein, 6 aa, Unknown
  Description of Unknown tumor-associated protease site sequence
PRWLRS

SEQ ID NO: 138 - protein, 8 aa, Unknown
  Description of Unknown tumor-associated protease site sequence
HPVGLLAR

SEQ ID NO: 139 - protein, 8 aa, Unknown
  Description of Unknown tumor-associated protease site sequence
KGPLGVRG

SEQ ID NO: 140 - protein, 5 aa, Unknown
  Description of Unknown tumor-associated protease site sequence
CGLDD

SEQ ID NO: 141 - protein, 8 aa, Unknown
  Description of Unknown tumor-associated protease site sequence
STAVIVSA

SEQ ID NO: 142 - protein, 8 aa, Unknown
  Description of Unknown tumor-associated protease site sequence
GPLGIAGQ

SEQ ID NO: 143 - protein, 6 aa, Unknown
  Description of Unknown tumor-associated protease site sequence
PLGLWA

SEQ ID NO: 144 - protein, 8 aa, Unknown
  Description of Unknown tumor-associated protease site sequence
GPLGMLSQ

SEQ ID NO: 145 - protein, 7 aa, Unknown
  Description of Unknown tumor-associated protease site sequence
PLGVRGK

SEQ ID NO: 146 - protein, 8 aa, Unknown
  Description of Unknown tumor-associated protease site sequence
GPQGIWGQ

SEQ ID NO: 147 - protein, 7 aa, Unknown
  Description of Unknown tumor-associated protease site sequence
AIPVSLR

SEQ ID NO: 148 - protein, 8 aa, Unknown
  Description of Unknown tumor-associated protease site sequence
GPLGLARK

SEQ ID NO: 149 - protein, 7 aa, Unknown
  Description of Unknown tumor-associated protease site sequence
PQGIAMG

SEQ ID NO: 150 - protein, 8 aa, Unknown
  Description of Unknown tumor-associated protease site sequence
VASSSTAV

SEQ ID NO: 151 - protein, 4 aa, Unknown
  Description of Unknown tumor-associated protease site sequence
PLGL

SEQ ID NO: 152 - protein, 8 aa, Unknown
  Description of Unknown tumor-associated protease site sequence
RPLALWRS

SEQ ID NO: 153 - protein, 8 aa, Unknown
  Description of Unknown tumor-associated protease site sequence
NKSRLGLG

SEQ ID NO: 154 - protein, 9 aa, Unknown
  Description of Unknown tumor-associated protease site sequence
GPQGIAGQR

SEQ ID NO: 155 - protein, 6 aa, Unknown
  Description of Unknown tumor-associated protease site sequence
PVGLIG

SEQ ID NO: 156 - protein, 8 aa, Unknown
  Description of Unknown tumor-associated protease site sequence
KPVSLSYR

SEQ ID NO: 157 - protein, 8 aa, Unknown
  Description of Unknown tumor-associated protease site sequence
QPVGINTS

SEQ ID NO: 158 - protein, 6 aa, Unknown
  Description of Unknown tumor-associated protease site sequence
PLGMTS

SEQ ID NO: 159 - protein, 5 aa, Unknown
  Description of Unknown tumor-associated protease site sequence
PRALM

SEQ ID NO: 160 - protein, 6 aa, Unknown
  Description of Unknown tumor-associated protease site sequence
GPLPLR

SEQ ID NO: 161 - protein, 8 aa, Unknown
  Description of Unknown tumor-associated protease site sequence
KQLRVVNG

SEQ ID NO: 162 - protein, 6 aa, Unknown
  Description of Unknown tumor-associated protease site sequence
PLGLYA

SEQ ID NO: 163 - protein, 7 aa, Unknown
  Description of Unknown tumor-associated protease site sequence
HSSKLQL

SEQ ID NO: 164 - protein, 5 aa, Unknown
  Description of Unknown tumor-associated protease site sequence
SSKYQ

SEQ ID NO: 165 - protein, 8 aa, Unknown
  Description of Unknown tumor-associated protease site sequence
GPLGVRGK

SEQ ID NO: 166 - protein, 6 aa, Unknown
  Description of Unknown tumor-associated protease site sequence
PRGMAS

SEQ ID NO: 167 - protein, 9 aa, Unknown
  Description of Unknown tumor-associated protease site sequence
AAALGNVAP

SEQ ID NO: 168 - protein, 9 aa, Unknown
  Description of Unknown tumor-associated protease site sequence
GGSGRSANA

SEQ ID NO: 169 - protein, 6 aa, Unknown
  Description of Unknown tumor-associated protease site sequence
PLGVRG

SEQ ID NO: 170 - protein, 8 aa, Unknown
  Description of Unknown tumor-associated protease site sequence
SGRSANAK

SEQ ID NO: 171 - protein, 9 aa, Unknown
  Description of Unknown tumor-associated protease site sequence
GPLGLWAGG

SEQ ID NO: 172 - protein, 5 aa, Unknown
  Description of Unknown tumor-associated protease site sequence
SGRSA

SEQ ID NO: 173 - protein, 6 aa, Unknown
  Description of Unknown tumor-associated protease site sequence
PLGLYL

SEQ ID NO: 174 - protein, 8 aa, Unknown
  Description of Unknown tumor-associated protease site sequence
SRRRVNSL

SEQ ID NO: 175 - protein, 5 aa, Unknown
  Description of Unknown tumor-associated protease site sequence
GSTFF

SEQ ID NO: 176 - protein, 8 aa, Unknown
  Description of Unknown tumor-associated protease site sequence
CPGRVVGG

SEQ ID NO: 177 - protein, 5 aa, Unknown
  Description of Unknown tumor-associated protease site sequence
GTQFF

SEQ ID NO: 178 - protein, 6 aa, Unknown
  Description of Unknown tumor-associated protease site sequence
PQGLAG

SEQ ID NO: 179 - protein, 5 aa, Unknown
  Description of Unknown tumor-associated protease site sequence
QVVAG

SEQ ID NO: 180 - protein, 8 aa, Unknown
  Description of Unknown tumor-associated protease site sequence
TYSRSRYL

SEQ ID NO: 181 - protein, 8 aa, Unknown
  Description of Unknown tumor-associated protease site sequence
NSGRAVTY

SEQ ID NO: 182 - protein, 8 aa, Unknown
  Description of Unknown tumor-associated protease site sequence
GPQGARGQ

SEQ ID NO: 183 - protein, 8 aa, Unknown
  Description of Unknown tumor-associated protease site sequence
PSSRRRVN

SEQ ID NO: 184 - protein, 7 aa, Unknown
  Description of Unknown tumor-associated protease site sequence
PLGLYAL

SEQ ID NO: 185 - protein, 8 aa, Unknown
  Description of Unknown tumor-associated protease site sequence
PMKRLTLG

SEQ ID NO: 186 - protein, 8 aa, Unknown
  Description of Unknown tumor-associated protease site sequence
DDDKIVGG

SEQ ID NO: 187 - protein, 8 aa, Unknown
  Description of Unknown tumor-associated protease site sequence
HLVEALYL

SEQ ID NO: 188 - protein, 8 aa, Unknown
  Description of Unknown tumor-associated protease site sequence
EVDLLIGS

SEQ ID NO: 189 - protein, 8 aa, Unknown
  Description of Unknown tumor-associated protease site sequence
AVRWLLTA

SEQ ID NO: 190 - protein, 5 aa, Unknown
  Description of Unknown tumor-associated protease site sequence
GGGRR

SEQ ID NO: 191 - protein, 24 aa, Artificial Sequence
  Description of Artificial Sequence Synthetic peptide
GGGSGGGSGGGSGGGSGGGSGGGS

SEQ ID NO: 192 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 193 - protein, 10 aa, Artificial Sequence
  Description of Artificial Sequence Synthetic peptide
  MISC_FEATURE (1)..(10): This sequence may encompass 5-10 residues
HHHHHHHHHH

(19) United States
(12) Patent Application Publication
(10) Pub. No.: US 2022/0281936 A1
(43) Pub. Date: Sep. 8, 2022

(54) COMPOSITIONS AND METHODS COMPRISING PROTEASE-ACTIVATED THERAPEUTIC AGENTS

(71) Applicant: The University of Chicago, Chicago, IL (US)

(72) Inventors: Jeffrey HUBBELL, Chicago, IL (US); Jun ISHIHARA, Chicago, IL (US); Juan MENDOZA, Chicago, IL (US); Aslan MANSUROV, Chicago, IL (US)

(73) Assignee: The University of Chicago, Chicago, IL (US)

(21) Appl. No.: 17/597,793

(22) PCT Filed: Jul. 24, 2020
(86) PCT No.: PCT/US2020/070308

§ 371 Date: Jan. 24, 2022

Related U.S. Application Data
(60) Provisional application No. 62/878,574, filed on Jul. 25, 2019.

(51) Int. Cl. C07K 14/54; C07K 14/715; A61K 47/68; A61K 39/395; A61P 35/00

(52) U.S. Cl. CPC C07K 14/5434; C07K 14/7155; A61K 47/6843; A61K 47/6851; A61K 39/3955; A61P 35/00; A61K 38/00

(57) ABSTRACT

he disclosure relates to the engineering of collagen-binding modification of masked therapeutic agents comprising one or more tumor-associated protease cleavage sites. Upon exposure to tumor-associated proteases in the tumor microenvironment, the polypeptide is cleaved, which unmasks the therapeutic agent, reducing off-target side effects and toxicity associated with systemic administration. Accordingly, aspects of the disclosure relate to a polypeptide comprising a therapeutic agent linked to a masking agent through a linker, wherein the linker comprises one or more tumor-associated protease cleavage sites, and wherein the masking agent blocks the association of the therapeutic agent to its therapeutic target, and further wherein the polypeptide is operatively linked to a collagen binding domain or a tumor-targeting agent.

### CROSS-REFERENCE TO RELATED APPLICATIONS

This application claims the benefit of priority of U.S. Provisional Patent Application No. 62/878,574 filed Jul. 25, 2019, which is hereby incorporated by reference in its entirety.

### BACKGROUND

Cytokine cancer immunotherapy using interleukin (IL)-12 has shown strong antitumor efficacy in both mouse and in human. However, due to its severe toxicity, some IL12 clinical trials have been terminated or unsuccessful. IL12 has not been approved to use in the clinic to date. Immunotherapies serve to activate immune responses, and as such, side-effects typically result from drug action in healthy organs. There is a need in the art for strategies to reduce the toxicity of therapeutic treatments.

### SUMMARY OF INVENTION

The disclosure relates to the engineering of collagen-binding modification of masked therapeutic agents comprising one or more tumor-associated protease cleavage sites.

Upon exposure to tumor-associated proteases in the tumor microenvironment, the polypeptide is cleaved, which unmasks the therapeutic agent, reducing off-target side effects and toxicity associated with systemic administration. Accordingly, aspects of the disclosure relate to a polypeptide comprising a therapeutic agent linked to a masking agent through a linker, wherein the linker comprises one or more tumor-associated protease cleavage sites, and wherein the masking agent blocks the association of the therapeutic agent to its therapeutic target, and further wherein the polypeptide is operatively linked to a collagen binding domain or a tumor-targeting agent.

Further aspects of the disclosure relate to a polypeptide comprising a cytokine linked to a masking agent through a linker, wherein the linker comprises one or more tumor-associated protease cleavage sites, and wherein the masking agent comprises a cytokine receptor polypeptide or fragment thereof that specifically binds to the cytokine. A masking agent refers to a molecule that blocks the association of a therapeutic agent with at least one binding partner. In some embodiments, the therapeutic agent comprises an antibody, and the binding partner comprises an antigen. In some embodiments, the therapeutic agent comprises a cytokine and the binding partner comprises a receptor polypeptide.

Further aspects relate to a composition comprising the polypeptides of the disclosure. Yet further aspects relate to nucleic acids encoding polypeptides of the disclosure and host cells comprising nucleic acids and/or polypeptides of the disclosure. Also provided are methods for making a polypeptide comprising expressing a nucleic acid of the disclosure in a host cell and isolating the expressed polypeptide. Further aspects relate to a method for treating cancer comprising administering a polypeptide or composition of the disclosure to a subject in need thereof, such as one that has cancer.

In some embodiments, the cytokine comprises interleukin-12 (IL12) and wherein the masking agent comprises interleukin 12 receptor (IL12R) polypeptide or an IL12-binding fragment thereof. In some embodiments, the IL12 comprises one or both of the p35 and p40 subunits. In some embodiments, the IL12 comprises the p35 and p40 subunits linked through a disulfide bond. In some embodiments, the IL12 comprises the p35 and p40 subunits linked through a peptide linker. In some embodiments, the IL12R polypeptide or fragment comprises interleukin 12 receptor beta 1 412101), or a fragment thereof. In some embodiments, the IL12R polypeptide or fragment comprises interleukin 12 receptor beta 2 412102), or a fragment thereof. In some embodiments, the IL12β1 polypeptide comprises one or both of fibronectin domains D1 and D2.

In some embodiments, the masking agent is fused to the N-terminus of the p35 subunit of IL12, and wherein the linker comprising the tumor-associated protease cleavage site is between the masking agent and the p35 subunit of IL12. In some embodiments, the masking agent is fused to the C-terminus of the p35 subunit of IL12, and wherein the linker comprising the tumor-associated protease cleavage site is between the masking agent and the p35 subunit of IL12. In some embodiments, the masking agent is fused to the C-terminus of the p40 subunit of IL12, and wherein the linker is between the masking agent and the p40 subunit of IL12. In some embodiments, the masking agent is fused to the N-terminus of the p40 subunit of IL12, and wherein the linker is between the masking agent and the p40 subunit of IL12. In some embodiments, the cytokine comprises interleukin-2 (IL-2) and the masking agent comprises interleukin 2 receptor alpha subunit (IL-2Rα), interleukin 2 receptor beta subunit (IL-2Rβ), interleukin 2 receptor gamma subunit (IL-2Rγ), fragments, or combinations of fragments thereof. In some embodiments, the cytokine comprises interferon-gamma (IFNγ) and the masking agent comprises interferon-gamma receptor 1 (IFNyR1), interferon-gamma receptor 2 (IFNγR2), fragments, or combinations of fragments thereof.

In some embodiments, the polypeptide comprises at least two tumor-associated protease cleavage sites. In some embodiments, the polypeptide comprises at least, at most, or exactly 1, 2, 3, 4, 5, 6, 7, or 8 tumor-associated protease cleavage sites, or any range derivable therein. In some embodiments, the tumor-associated protease cleavage site comprises a uPA, matrix metalloproteinase, or thrombin cleavage site. In some embodiments, the tumor-associated protease cleavage site comprises at least one tumor associated protease cleavage site described herein. In some embodiments, the tumor-associated cleavage site comprises a cleavage site with an amino acid of one of SEQ ID NOS:13, 14, 49, 51, 55, 109-190. In some embodiments, the polypeptide comprises at least two different tumor-associated protease cleavage site. In some embodiments, the polypeptide comprises at least 2, 3, or 4 different tumor-associated protease cleavage sites, or any range derivable therein. In some embodiments, the polypeptide comprises at least 2 of the same tumor-associated protease cleavage sites. In some embodiments, the polypeptide comprises at least 2, 3, 4, 5, 6, 7, or 8 of the same protease cleavage sites, or any range derivable therein. In embodiments which comprise more than one protease cleavage site, the protease cleavage sites may be adjacent or may have intervening amino acids. In some embodiments, at least, at most, or exactly 0, 1, 2, 3, 4, 5, 6, 7, 8, 9, 10, 11, 12, 13, 14, 15, 16, 17, 18, 19, or 20 amino acids, or any range derivable therein, separate one tumor-associated protease cleavage site from another tumor-associated protease cleavage site.

In some embodiments, the cytokine comprises an anti-inflammatory cytokine. In some embodiments, the cytokine comprises a pro-inflammatory cytokine.

In some embodiments, the polypeptide is conjugated to a tumor targeting agent. In some embodiments, the tumor targeting agent comprises an antibody or an antigen-binding fragment thereof. In some embodiments, the antibody or antigen-binding fragment comprises a stroma targeting antibody or stroma-binding fragment thereof. In some embodiments, the antibody or binding fragment specifically binds to fibronectin, alternatively spliced domains of fibronectin, collagens, tenascins, periostins, syndecans, proteoglycans, or a tumor stroma cell-specific antigen. In some embodiments, the antibody or binding fragment specifically binds toe extra domain A (EDA) or extra domain B (EDB) of fibronectin. In some embodiments, the tumor targeting agent comprises a Fab that specifically binds to an alternatively spliced domain of fibronectin comprising extra domain A (EDA). In some embodiments, the tumor targeting agent comprises an antibody or antigen binding fragment thereof that specifically binds to a tumor-associated antigen. Other tumor targeting agents include those recited in US20140294723A1, WO2001062298A2, WO1997045544A1, WO2006119897A2, WO2006050834A2, WO2008120101A2, WO2010078916A1, which are herein incorporated by reference.

In some embodiments, the tumor targeting agent comprises a collagen binding domain. In some embodiments, the polypeptide comprises at least two collagen binding domains. In some embodiments, the polypeptide comprises at least 2, 3, 4, 5, or 6 collagen binding domains. In some embodiments, the polypeptide comprises a collagen binding domain from decorin or von Willebrand factor (VWF).

In some embodiments, the polypeptide further comprises a serum protein conjugated to the polypeptide. In some embodiments, the serum protein is conjugated to the polypeptide through a peptide bond. In some embodiments, the serum protein comprises albumin or a fragment thereof. In some embodiments, the serum protein is at least 40, 45, 50, 55, 60, 65, 70, or 75 kDa (or any range derivable therein).

In some embodiments, the polypeptide comprises a second linker. In some embodiments, the second linker comprises glycine and serine amino acid residues. In some embodiments, the polypeptide comprises a third, fourth, or fifth linker. In some embodiments, the third, fourth, or fifth linker comprises glycine and serine amino acid residues. In some embodiments, the linker comprises (GGGS)ₙ, (SEQ ID NO:48) wherein n=1, 2, 3, 4, 5, 6, 7, or 8, or any range derivable therein, or GGGSGGGS (SEQ ID NO:47). In some embodiments, the second linker comprises (GGGS)ₙ (SEQ ID NO:48), wherein n=6. In some embodiments, the polypeptide comprises a protein tag. In some embodiments, the protein tag comprises a 6H tag. In some embodiments, the protein tag comprises a protein tag described herein. In some embodiments, the polypeptide is not operatively linked to a particle, nanovesicle, or liposome. In some embodiments, the composition does not comprise a liposome, particle, or nanovescicle.

In some embodiments, the methods or the disclosure relate to the treatment of skin cancer, such as for the treatment of melanoma. In some embodiments, methods of the disclosure further comprise administration of one or more additional cancer therapies. In some embodiments, the additional therapy is one described herein. In some embodiments, the subject has or will receive an immunotherapy. In some embodiments, the method further comprises administration of an immunotherapy. In a particular embodiment, the immunotherapy comprises an immune checkpoint inhibitor. The immune checkpoint inhibitor may be an anti-PD-1 monoclonal antibody or an anti-CTLA-4 monoclonal antibody. Further exemplary immune checkpoint proteins that may be inhibited in embodiments of the disclosure are described herein. In some embodiments, the immune checkpoint inhibitor comprises one or more of nivolumab, pembrolizumab, pidilizumab, ipilimumab or tremelimumab. In some embodiments, the immune checkpoint therapy is monotherapy. The term monotherapy, in the context of immune checkpoint therapy, refers to administration of one immune checkpoint inhibitor during the course of therapy. The monotherapy may be a therapy comprising of only one of a PD-1, PDL1, PDL2, CTLA-4, B7-1, or B7-2 inhibitor. In some embodiments, the immune checkpoint inhibitor therapy comprises combination therapy. For example, the combination therapy may be a combination of (i) a PD-1, PDL1, or PDL2 inhibitor and (ii) a CTLA-4, B7-1, or B7-2 inhibitor. Particular combination therapies include those that comprise an anti-PD-1 antibody and an anti-CTLA-4 antibody. Further immunotherapies useful in the methods and compositions of the disclosure are described herein. In some embodiments, the immunotherapy or additional therapy is administered before, after, or concurrent with the polypeptide. In some embodiments, the polypeptide or composition is administered systemically. In some embodiments, the polypeptide or composition is administered by a route of administration described herein. In some embodiments, the polypeptide or composition is administered by intravenous injection. In some embodiments, the subject has been previously treated with a cancer therapy. In some embodiments, the subject has been determined to be non-responsive to the previous treatment or wherein the wherein the subject experienced non-specific toxicity to the previous treatment.

The term “cytokine polypeptide” as used herein refers to a polypeptide, which is cytokine or a receptor binding domain thereof and retains at a portion of cytokine activity.

The terms “protein”, “polypeptide” and “peptide” are used interchangeably herein when referring to a gene product comprising a polymer of amino acids.

The terms “subject,” “mammal,” and “patient” are used interchangeably. In some embodiments, the subject is a mammal. In some embodiments, the subject is a human. In some embodiments, the subject is a mouse, rat, rabbit, dog, donkey, or a laboratory test animal such as fruit fly, zebrafish, etc.

It is contemplated that the methods and compositions include exclusion of any of the embodiments described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C. Schematic of fusion of the IL12Rβ1 recombinant protein to IL12.

(A) Schematic of fusion of the IL12Rβ1 recombinant protein to IL12. IL12Rβ1-IL12 receptor binding site is not exposed in the peripheral tissues, but tumor specific protease exposes IL12 binding site within tumor. (B-C) Structure of IL12Rβ1 fibronectin I and II domains fusion to IL12.

FIG. 2A-B. IL12Rβ1 fibronectin I and II domains fusion to IL12 inactivates IL12 activity in vitro and in vivo. (A) IFNγ concentration after splenocyte culture in vitro. After 2 days of culture in the presence of IL12, IFNγ concentration in supernatant was measured by ELISA. (B) 5×10⁵ B16F10 cells were inoculated on day 0. IL12 variants (25 μg, IL12 basis) was injected i.v. on day 7. On day 9, blood was collected and IFNγ concentration in blood serum was determined by ELISA.

FIG. 3A-P. IL12Rβ1 fusion reduces potential treatment-related toxicity of IL12. (A) In vitro cleavage of IL12Rβ1-VP-IL12 and of IL12Rβ1-LS-IL12 by MMPs and uPA. IL-12, IL12Rβ1-VP-IL12 and IL12Rβ1-LS-IL12 were either treated with assay buffer only (Blank), MMP2, MMP9 or uPA for 30 min at 37° C. Decrease of molecular weight from —105 kDa to —60 kDa indicates proteolytic cleavage of IL12Rβ1-VP-IL12 and IL12Rβ1-LS-IL12. (B) In vitro activity of IL12Rβ1-VP-IL12, IL12Rβ1-LS-IL12 and the non-cleavable of IL12Rβ1-(G₃S)₁₁-IL12. IL-12, IL12Rβ1-VP-IL12, IL12Rβ1-LS-IL12 and IL12Rβ1-(G3S)₁₁-IL12 were applied on pre-activated mouse CD8⁺ T cells at indicated concentrations (n=2 per condition) and phosphorylation of STAT4 was assessed by flow cytometry. Dose-response relationship and half-maximal activation values are shown. (C) In vitro activity of IL12Rβ1-VP-IL12, IL12Rβ1-LS-IL12 after treatment with proteases. IL12Rβ1-VP-IL12 or IL12Rβ1-LS-IL12 were first treated with MMP2 or uPA, respectively. Cleaved constructs or IL12 were applied on pre-activated mouse CD8⁺ T cells at indicated concentrations (n=2 per condition) and phosphorylation of STAT4 was assessed by flow cytometry. Dose-response relationship and half-maximal activation values are shown. (D) In vivo toxicity of IL12Rβ1-VPLS-IL12-CBD in healthy mice. C57BL/6 mice were treated i.v. with either PBS, IL12 or IL12Rβ1-VPLS-IL12-CBD (doses are indicated on IL12 molar basis) on days 0, 3 and 6. On days 2, 5 and 8, mice were bled and sera were analyzed for the presence of proinflammatory cytokines using LEGENDplex cytokine release syndrome panel. (E) White blood cell count and (F) platelets count were measured by hematology analyzer. (G-N) Blood toxicity markers were analyzed after IL12Rβ1-IL12 injection to non-tumor bearing mice. The graphs depict an analysis of liver damage markers (blood albumin concentration, total protein, alanine aminotransferase (ALT) activity, aspartate aminotransferase (AST) activity, and alkaline phosphate activity), kidney damage marker (total bilirubin), pancreas damage marker (amylase), and lung damage marker (CO2 concentration). (O-P) Blood toxicity markers (ALT activity and amylase) were analyzed after IL12Rβ1-IL12 and anti-PD-1 antibody injection to B16F10 tumor bearing mice. Statistical analyses were done using ANOVA with Tukey's test. *p<0.05**p<0.01; N.S.=not significant.

FIG. 4A-D. IL12Rβ1-IL12 with cleavable linker treatment reduces growth rate of B16F10 melanoma. 5×10⁵ B16F10 cells were inoculated on day 0. IL12 (25 μg), equimolar IL12 variants or PBS was administered i.v. on (A) day 8 or (B) day 7. (A-B) IL12β1-IL12 with uPA protease cleavable linker (LS) was used. (C) IL12 (5 μg) and IL12R1-IL12 (50 μg) with uPA and MMP protease cleavable linker (VP-LS) were injected i.v. from day 7, every 3 days. (D) 100 μg of IL12R1-IL12 with cleavable linkers (HP, VP, and LS) were injected i.v. on days 7 and 10. Anti-PD-1 antibody was injected i.p. on days 7, 10, and/or 13. Graphs depict tumor volume until the first mouse died. Tumor volumes are presented as mean ±SEM. n=3-4

### DETAILED DESCRIPTION

Cytokines are key factors for antitumor activities, but not many of them have been translated to the clinic to date. IL12 is one of the strongest antitumor cytokines, but due to its high toxicity, the clinical trial has been terminated or unsuccessful. Thus, decreasing its toxicity is an important strategy to translate it to the clinic. To improve CBD-IL12 therapy, a domain of the IL12 receptor IL12Rβ1 was fused to the IL12, to form IL12Rβ1-IL12. This fusion is inactive, but the inclusion of an MMP or thrombin cleavage site between the receptor making agent and the cytokine yields a pro-cytokine that can be activated in the tumor microenvironment. The inventors have demonstrated that the immunotoxicity of the IL12 is thus reduced, and that the IL12Rβ1-IL12 fusion with the protease-sensitive linker retains therapeutic utility. The inventors also found that introducing multi-cleavage sites in the linker (e.g. tandem MMP, tandem thrombin, and MMP-thrombin dyads and repeats) would increase the protease sensitivity and may increase the antitumor efficacy of IL12Rβ1-IL12 therapy.

Furthermore, the use of a collagen binding domain fused to the masked therapeutic molecules of the disclosure is particularly useful, since the CBD increases the retention of the masked therapeutic agent in the tumor microenvironment, which prolongs the exposure of the masked therapeutic agent to the protease and increases the local concentration of the unmasked therapeutic agent. In conclusion, the inventors have developed a technology to reduce toxicity of therapeutic agents by fusing the cytokine receptor to the cytokine. Tumor specific proteases cleave the linker to activate the cytokine within the tumor.

### I. Polypeptide Embodiments

A. Therapeutic Agents and Masking Agents

Embodiments of the disclosure relate to therapeutic agent and masking agents that bind to the therapeutic agent and prevent association of the therapeutic agent with its target to reduce toxicity associated with the therapeutic agent. The polypeptides of the disclosure comprise a tumor-associated protease cleavage site that unmasks the therapeutic agent when it encounters the relevant protease. Since the protease is one that is enriched in the tumor microenvironment, there is a reduction of the active therapeutic agent in normal tissues when administered systemically, compared to the systemic administration of the unmasked therapeutic agent.

1. Cytokines

In some embodiments, the therapeutic agent comprises a cytokine or a therapeutic polypeptide from a cytokine. In certain embodiments, the cytokine comprises a functionally active fragment of a cytokine. In some embodiments, the functionally active cytokine fragment binds and activates the corresponding receptor. In some embodiments, the cytokine comprises IL12. IL12 is a heterodimeric glycosylated cytokine comprised of disulfide-linked p35 (-35 kDa) and p40 (˜40 kDa) subunits. The human IL12 p35 sequence is represented by the following:

RNLPVATPDPGMFPCLHHSQNLLRAVSNMLQKARQTLEFYPCT SEEIDHEDITKDKT STVEACLPLELTKNESCLNSRETSFITNGSCLASRKTSFMMALCLSSIYEDLKMYQVE FKTMNAKLLMDPKRQIFLDQNMLAVIDELMQALNENSETVPQKS SLEEPDFYKTKIK LCILLHAFRIRAVTIDRVMSYLNAS (SEQ ID NO:3). The human IL12 p40 sequence is represented by the following: IWELKKDVYVVELDWYPDAPGEMVVLTCDTPEEDGITWTLDQSSEVLGSGKTLTIQ VKEFGDAGQYTCHKGGEVLSHSLLLLHKKEDGIWSTDILKDQKEPKNKTFLRCEAK

NYSGRETCWWLTTISTDLTESVKSSRGSSDPQGVTCGAATLSAERVRGDNKEYEYSV ECQEDSACPAAEESLPIEVMVDAVHKLKYENYT S SFFIRDIIKPDPPKNLQLKPLKNSR QVEVSWEYPDTWSTPHSYFSLTFCVQVQGKSKREKKDRVETDKTSATVICRKNASIS VRAQDRYYSSSWSEWASVPCS (SEQ ID NO:4). The mouse IL12 p35 sequence is represented by the following:

RVIPVSGPARCLSQSRNLLKTTDDMVKTAREKLKHYSCTAEDIDHEDITRDQTSTLKT CLPLELHKNESCLATRET S STTRGSCLPPQKTSLMMTLCLGSIYEDLKMYQTEFQAIN AALQNHNHQQIILDKGMLVAIDELMQ SLNHNGETLRQKPPVGEADPYRVKMKLCIL LHAFSTRVVTINRVMGYLSSA (SEQ ID NO:5). The mouse IL12 p40 sequence is represented by the following:

(SEQ ID NO: 6)
MWELEKDVYVVEVDWTPDAPGETVNLTCDTPEEDDITWTSDQRHGVIGSG
KTLTITVKEFLDAGQYTCHKGGETLSHSHLLLHKKENGIWSTEILKNFKN
KTFLKCEAPNYSGRFTCSWLVQRNMDLKFNIKSSSSSPDSRAVTCGMASL
SAEKVTLDQRDYEKYSVSCQEDVTCPTAEETLPIELALEARQQNKYENYS
TSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSS
CSKWACVPCRVRS.

Suitable IL12 masking agents include polypeptides that bind to IL12 and prevent binding of IL12 to other molecules, such as IL12R. Exemplary polypeptides include polypeptides from IL12R, such as IL12Rf31 and IL12Rβ2.

The mouse IL12Rβ1 is represented by a polypeptide with either of the following amino acid sequences:

(SEQ ID NO: 2)
QLGASGPGDGCCVEKTSFPEGASGSPLGPRNLSCYRVSKTDYECSWQYDG
PEDNVSHVLWCCFVPPNHTHTGQERCRYFSSGPDRTVQFWEQDGIPVLSK
VNFWVESRLGNRTMKSQKISQYLYNWTKTTPPLGHIKVSQSHRQLRMDWN
VSEEAGAEVQFRRRMPTTNWTLGDCGPQVNSGSGVLGDIRGSMSESCLCP
SENMAQEIQIRRRRRLSSGAPGGPWSDWSMPVCVPPEVLPQALVPRGS
and
(SEQ ID NO: 20)
QLGASGPGDGCCVEKTSFPEGASGSPLGPRNLSCYRVSKTDYECSWQYDG
PEDNVSHVLWCCFVPPNHTHTGQERCRYFSSGPDRTVQFWEQDGIPVLSK
VNFWVESRLGNRTMKSQKISQYLYNWTKTTPPLGHIKVSQSHRQLRMDWN
VSEEAGAEVQFRRRMPTTNWTLGDCGPQVNSGSGVLGDIRGSMSESCLCP
SENMAQEIQIRRRRRLSSGAPGGPWSDWSMPVCVPPEVLPQAKIKFLVEP
LNQGGRRRLTMQGQSPQLAVPEGCRGRPGAQVKKHLVLVRMLSCRCQAQT
SKTVPLGKKLNLSGATYDLNVLAKTRFGRSTIQKWHLPAQELTETRALNV
SVGGNMTSMQWAAQAPGTTYCLEWQPWFQHRNHTHCTLIVPEEEDPAKMV
THSWSSKPTLEQEECYRITVFASKNPKNPMLWATVLSSYYFGGNASRAGT
PRHVSVRNQTGDSVSVEWTASQLSTCPGVLTQYVVRCEAEDGAWESEWLV
PPTKTQVTLDGLRSRVMYKVQVRADTARLPGAWSUPQRFSFEVQISRLSI
IFASLGSFASVLLVGSLGYIGLNRAAWHLCPPLPTPCGSTAVEFPGSQGK
QAWQWCNPEDFPEVLYPRDALVVEMPGDRGDGTESPQAAPECALDTRRPL
ETQRQRQVQALSEARRLGLAREDCPRGDLAHVTLPLLLGGVTQGASVLDD
LWRTHKTAEPGPPTLGQEA.

The human IL12Rβ1 is represented by a polypeptide with the following amino acid sequence:

(SEQ ID NO: 19)
CRTSECCFQDPPYPDADSGSASGPRDLRCYRISSDRYECSWQYEGPTAGV
SHFLRCCLSSGRCCYFAAGSATRLQFSDQAGVSVLYTVTLWVESWARNQT
EKSPEVTLQLYNSVKYEPPLGDIKVSKLAGQLRMEWETPDNQVGAEVQFR
HRTPSSPWKLGDCGPQDDDTESCLCPLEMNVAQEFQLRRRQLGSQGSSWS
KWSSPVCVPPENPPQPQVRFSVEQLGQDGRRRLTLKEQPTQLELPEGCQG
LAPGTEVTYRLQLHMLSCPCKAKATRTLHLGKMPYLSGAAYNVAVISSNQ
FGPGLNQTWHIPADTHTEPVALNISVGTNGTTMYWPARAQSMTYCIEWQP
VGQDGGLATCSLTAPQDPDPAGMATYSWSRESGAMGQEKCYYITIFASAH
PEKLTLWSTVLSTYHFGGNASAAGTPHHVSVKNHSLDSVSVDWAPSLLST
CPGVLKEYVVRCRDEDSKQVSEHPVQPTETQVTLSGLRAGVAYTVQVRAD
TAWLRGVWSQPQRFSIEVQVSDWLIFFASLGSFLSILLVGVLGYLGLNRA
ARHLCPPLPTPCASSAIEFPGGKETWQWINPVDFQEEASLQEALVVEMSW
DKGERTEPLEKTELPEGAPELALDTELSLEDGDRCKAKM.

The human IL12Rβ2 is represented by a polypeptide with the following amino acid sequence:

KIDACKRGDVTVKP SHVILLGSTVNITCSLKPRQGCFHYSRRNKLILYKFDRRINFHH GHSLNSQVTGLPLGTTLFVCKLACINSDEIQICGAEIFVGVAPEQPQNLSCIQKGEQGT VACTWERGRDTHLYTEYTLQLSGPKNLTWQKQCKDIYCDYLDFGINLTPESPESNFT AKVTAVNSLGS S S SLP STFTFLDIVRPLPPWDIRIKFQKASVSRCTLYWRDEGLVLLN RLRYRPSNSRLWNMVNVTKAKGRHDLLDLKPFTEYEFQISSKLHLYKGSWSDWSES

LRAQTPEEEPTGMLDVWYMKRHIDYSRQQISLFWKNL SV SEARGKILHYQVTLQELT GGKAMTQNITGHTSWTTVIPRTGNWAVAVSAANSKGS SLPTRINIMNLCEAGLLAPR QVSANSEGMDNILVTWQPPRKDP SAVQEYVVEWRELHPGGDTQVPLNWLRSRPYN VSALISENIK SYICYEIRVYAL SGDQGGC S SILGNSKHKAPLSGPHINAITEEKGSILISW NSIPVQEQMGCLLHYRIYWKERDSNSQPQLCEIPYRVSQNSHPINSLQPRVTYVLWM

TALTAAGESSHGNEREFCLQGKANWMAFVAPSICIAIIIVIVGIFSTHYFQQKVFVLLAA LRPQWC SREIPDPANSTCAKKYPIAEEKTQLPLDRLLIDWPTPEDPEPLVISEVLHQVT PVFRUPPC SNWPQREKGIQGHQASEKDMMHSAS SPPPPRALQAESRQLVDLYKVLES RGSDPKPENPACPWTVLPAGDLPTHDGYLP SNIDDLP SHEAPLAD SLEELEPQHISL S VFPSSSLHPLTFSCGDKLTLDQLKMRCDSLML (SEQ ID NO:21). The mouse IL12102 is represented by a polypeptide with the following amino acid sequence:

(SEQ ID NO: 22)
NIDVCKLGTVTVQPAPVIPLGSAANISCSLNPKQGCSHYPSSNELILLKF
VNDVLVENLHGKKVHDHTGHSSTFQVTNLSLGMTLFVCKLNCSNSQKKPP
VPVCGVEISVGVAPEPPQNISCVQEGENGTVACSWNSGKVTYLKTNYTLQ
LSGPNNLTCQKQCFSDNRQNCNRLDLGINLSPDLAESRFIVRVTAINDLG
NSSSLPHTFTFLDIVIPLPPWDIRINFLNASGSRGTLQWEDEGQVVLNQL
RYQPLNSTSWNMVNATNAKGKYDLRDLRPFTEYEFQISSKLHLSGGSWSN
WSESLRTRTPEEEPVGILDIWYMKQDIDYDRQQISLFWKSLNPSEARGKI
LHYQVTLQEVTKKTTLQNTTRHTSWTRVIPRTGAWTASVSAANSKGASAP
THINIVDLCGTGLLAPHQVSAKSENMDNILVTWQPPKKADSAVREYIVEW
RALQPGSITKFPPHWLRIPPDNMSALISENIKPYICYEIRVHALSESQGG
CSSIRGDSKHKAPVSGPHITAITEKKERLFISWTHIPFPEQRGCILHYRI
YWKERDSTAQPELCEIQYRRSQNSHPISSLQPRVTYVLWMTAVTAAGESP
QGNEREFCPQGKANWKAFVISSICIAIITVGTFSIRYFRQKAFTLLSTLK
PQWYSRTIPDPANSTWVKKYPILEEKIQLPTDNLLMAWPTPEEPEPLIIH
EVLYHMIPVVRQPYYFKRGQGFQGYSTSKQDAMYIANPQATGTLTAETRQ
LVNLYKVLESRDPDSKLANLTSPLTVTPVNYLPSHEGYLPSNIEDLSPHE
ADPTDSFDLEHQHISLSIFASSSLRPLIFGGERLTLDRLKMGYDSLMSNE
A.

In some embodiments, the cytokine comprises a polypeptide comprising an amino acid sequence of SEQ ID NO:3-6, or a polypeptide comprising an amino acid sequence of a fragment of the polypeptides represented by SEQ ID NO:3-6, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:3-6 or a fragment thereof

In some embodiments, the cytokine comprises an IL12 polypeptide and the masking agent comprises an amino acid sequence of SEQ ID NO:2, or 19-22, or a polypeptide comprising an amino acid sequence of a fragment of the polypeptides represented by SEQ ID NO:2, or 19-22, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:2, or 19-22 or a fragment thereof.

In some embodiments, the cytokine comprises IL-2. The human IL-2 sequence comprises MAPT S S S TKKTQLQLEHLLLDLQMILNGINNYKNPKLTRMLTFKFYMPKKATELKHL QCLEEELKPLEEVLNLAQSKNFHLRPRDLISNINVIVLELKGSETTFMCEYADETATIV EFLNRWITFAQSIISTLT (SEQ ID NO:23). The mouse IL-2 sequence comprises PTSSSTSSSTAEAQQQQQQQQQQQQHLEQLLMDLQELLSRMENYRNLKLPRMLTFK

FYLPKQATELKDLQCLEDELGPLRHVLDLTQ SK SFQLEDAENFISNIRVTVVKLKGSD NTFECQFDDESATVVDFLRRWIAFCQSIISTSPQ (SEQ ID NO:24).

In some embodiments, the masking agent for IL-2 comprises an IL-2R polypeptide. In some embodiments, the IL-2R polypeptide comprises a polypeptide from the IL-2Rbeta, IL-2Ralpha, or IL-2Rgamma subunit. Human Interleukin-2 receptor subunit beta comprises the following amino acid sequence:

(SEQ ID NO: 27)
AVNGTSQFTCFYNSRANISCVWSQDGALQDTSCQVHAWPDRRRWNQTCEL
LPVSQASWACNLILGAPDSQKLTTVDIVTLRVLCREGVRWRVIVIAIQDF
KPFENLRLMAPISLQVVHVETHRCNISWEISQASHYFERHLEFEARTLSP
GHTWEEAPLLTLKQKQEWICLETLTPDTQYEFQVRVKPLQGEFTTWSPWS
QPLAFRTKPAALGKDT.

Mouse Interleukin-2 receptor subunit beta has the following amino acid sequence:

(SEQ ID NO: 28)
AVKNCSHLECFYNSRANVSCMWSHEEALNVTTCHVHAKSNLRHWNKTCEL
TLVRQASWACNLILGSFPESQSLTSVDLLDINVVCWEEKGWRRVKTCDFH
PFDNLRLVAPHSLQVLHIDTQRCNISWKVSQVSHYIEPYLEFEARRRLLG
HSWEDASVLSLKQRQQWLFLEMLIPSTSYEVQVRVKAQRNNTGTWSPWSQ
PLTFRTRPADPMKE.

Human Interleukin-2 receptor subunit alpha has the following amino acid sequence: ELCDDDPPEIPHATFKAMAYKEGTMLNCECKRGFRRIKSGSLYMLCTGNS SHSSWD NQCQCTS SATRNTTKQVTPQPEEQKERKTTEMQSPMQPVDQASLPGHCREPPPWEN EATERIYHFVVGQMVYYQCVQGYRALHRGPAESVCKMTHGKTRWTQPQLICTGEM

ETSQFPGEEKPQASPEGRPESETSCLVTTTDFQIQTEMAATMETSIFTTEYQ (SEQ ID

NO:29). Mouse Interleukin-2 receptor subunit alpha has the following amino acid sequence:

(SEQ ID NO: 30)
ELCLYDPPEVPNATFKALSYKNGTILNCECKRGERRLKELVYMRCLGNSW
SSNCQCTSNSHDKSRKQVTAQLEHQKEQQTTTDMQKPTQSMHQENLTGHC
REPPPWKHEDSKRIYHFVEGQSVHYECIPGYKALQRGPAISICKMKCGKT
GWTQPQLTCVDEREHERFLASEESQGSRNSSPESETSCPITTTDEPQPTE
TTAMTETFVLTMEYK.

Human Interleukin-2 receptor subunit gamma has the following amino acid sequence: LNTTILTPNGNEDTTADFFLTTMPTD SL S VSTLPLPEVQCF VFNVEYMNCTWNS S SEP QPTNLTLHYWYKNSDNDKVQKC SHYLF SEEITSGCQLQKKEIHLYQTFVVQLQDPRE PRRQATQMLKLQNLVIPWAPENLTLHKLSESQLELNWNNRFLNHCLEHLVQYRTDW

DHSWTEQ SVDYRHKF SLP S VDGQKRYTFRVRSRFNPLCGSAQHW SEW SHPIHWGSN TSKENPFLFALEA (SEQ ID NO:31). Mouse Interleukin-2 receptor subunit gamma has the following amino acid sequence:

(SEQ ID NO: 32)
WSSKVLMSSANEDIKADLILTSTAPEHLSAPTLPLPEVQCFVFNIEYMNC
TWNSSSEPQATNLTLHYRYKVSDNNTFQECSHYLFSKEITSGCQIQKEDI
QLYQTFVVQLQDPQKPQRRAVQKLNLQNLVIPRAPENLTLSNLSESQLEL
RWKSRHIKERCLQYLVQYRSNRDRSWTELIVNHEPRFSLPSVDELKRYTF
RVRSRYNPICGSSQQWSKWSQPVHWGSHTVEENPSLFALEA.

In some embodiments, the cytokine comprises a polypeptide comprising an amino acid sequence of SEQ ID NO:23 or 24, or a polypeptide comprising an amino acid sequence of a fragment of the polypeptides represented by SEQ ID NO:23 and 24, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:23 or 24 or a fragment thereof.

In some embodiments, the cytokine comprises an IL-2 polypeptide and the masking agent comprises an amino acid sequence of SEQ ID NO:27-32, or a polypeptide comprising an amino acid sequence of a fragment of the polypeptides represented by SEQ ID NO:27-32, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:27-32 or a fragment thereof.

In some embodiments, the cytokine comprises IFNy. The mouse IFNγ comprises the following sequence: HGTVIESLESLNNYFNS SGIDVEEKSLFLDIWRNWQKDGDMKILQSQIISFYLRLFEVL KDNQAISNNIS VIESHLITTFF SNSKAKKDAFMSIAKFEVNNPQVQRQAFNELIRVVHQ LLPESSLRKRKRSRC (SEQ ID NO:25). The human IFNγ comprises the following sequence: QDPYVKEAENLKKYFNAGHSDVADNGTLFLGILKNWKEESDRKIMQ SQIV SFYFKLF KNFKDDQSIQKSVETIKEDMNVKFFNSNKKKRDDFEKLTNYSVTDLNVQRKAIHELI QVMAELSPAAKTGKRKRSQMLFQGRRASQ (SEQ ID NO:26). The IFNγ may be a functional fragment, such as one that is truncated at the C-terminus. For example, the IFNy polypeptide may be one comprising at least 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 contiguous amino acids of SEQ ID NO:25 or 26. In some embodiments, the IFNy polypeptide may be one comprising at least amino acids 1 to 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 of SEQ ID NO:25 or 26 (or any range derivable therein).

In some embodiments, the masking agent for a IFNγ polypeptide comprises a polypeptide from the IFNγ receptor 1 or IFNγ receptor 2. The human IFNγ receptor 1 comprises the following sequence:

EMGTADLGP S S VPTPTNVTIESYNMNPIVYWEYQIMPQ VPVFTVEVKNYGVKNSEWI DACINISHHYCNISDHVGDP SNSLWVRVKARVGQKESAYAKSEEFAVCRDGKIGPPK LDIRKEEKQIMIDIFHP SVFVNGDEQEVDYDPETTCYIRVYNVYVRMNGSEIQYKILT QKEDDCDEIQCQLAIPVS SLNSQYC VSAEGVLHVWGVTTEKSKEVCITIFNS SIKG

(SEQ ID NO:33). The mouse IFNγ receptor 1 comprises the following sequence: ALTSTEDPEPPSVPVPTNVLIKSYNLNPVVCWEYQNMSQTPIFTVQVKVYSGSWTDS CTNISDHCCNIYEQIIVIYPDVSAWARVKAKVGQKESDYARSKEFLMCLKGKVGPPGL EIRRKKEEQL SVLVFHPEVVVNGESQGTMFGDGSTCYTFDYTVYVEHNRSGEILHTK HTVEKEECNETLCELNISVSTLDSRYCISVDGISSFWQVRTEKSKDVCIPPFHDDRKDS (SEQ ID NO:34). The human IFNγ receptor 2 comprises the following sequence: SQLPAPQHPKIRLYNAEQVLSWEPVALSNSTRPVVYQVQFKYTDSKWFTADIMSIGV NCTQITATECDFTAASPSAGFPMDFNVTLRLRAELGALHSAWVTMPWFQHYRNVTV GPPENIEVTPGEGSLIIRFSSPFDIADTSTAFFCYYVHYWEKGGIQQVKGPFRSNSISLD

NLKPSRVYCLQVQAQLLWNKSNIFRVGHLSNISCYETMADASTELQQ (SEQ ID

NO:35). The mouse IFNγ receptor 2 comprises the following sequence:

(SEQ ID NO: 36)
ASSPDSFSQLAAPLNPRLHLYNDEQILTWEPSPSSNDPRPVVYQVEYSFI
DGSWHRLLEPNCTDITETKCDLTGGGRLKLFPHPFTVFLRVRAKRGNLTS
KWVGLEPFQHYENVTVGPPKNISVTPGKGSLVIHFSPPFDVFHGATFQYL
VHYWEKSETQQEQVEGPFKSNSIVLGNLKPYRVYCLQTEAQLILKNKKIR
PHGLLSNVSCHETTANASARLQQVILIPLGIFALLLGLTGACFTLFLKYQ
SRVKYWFQAPPNIPEQIEEYLKDPDQFILEVLDKDGSPKEDSWDSVSIIS
SPEKERDDVLQTP.

In some embodiments, the cytokine comprises a polypeptide comprising an amino acid sequence of SEQ ID NO:25 or 26, or a polypeptide comprising an amino acid sequence of a fragment of the polypeptides represented by SEQ ID NO:25 and 26, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:25 or 26 or a fragment thereof.

In some embodiments, the cytokine comprises an IFNγ polypeptide and the masking agent comprises an amino acid sequence of SEQ ID NO:33-36, or a polypeptide comprising an amino acid sequence of a fragment of the polypeptides represented by SEQ ID NO:33-36, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:33-36 or a fragment thereof

In embodiments of the disclosure, the masking agent made be a polypeptide or a functional fragment of a polypeptide described herein. In some embodiments, the masking agent comprises a receptor polypeptide or a fragment thereof that binds to the cytokine.

2. Antibodies

In some embodiments, the therapeutic agent comprises an antibody, such as a therapeutic antibody. In some embodiments, the therapeutic antibody is modified by the site-specific substitution of an amino acid in either the heavy or light chain variable region with a cysteine (Cys). The sulfhydryl (SH) group in the side chain of the substituted-in Cys serves as a chemical handle for attaching a masking agent that interferes with the antibody's ability to bind to its antigen. The masking agent may be a group that sterically inhibits antibody-antigen binding, but otherwise does not specifically interact with either the antibody or the antigen.

Alternatively, the masking agent can interact with the antibody, for example by electrostatic or van der Waals forces. A tumor-associated protease cleavage site may be between or link the masking agent and the antibody.

In some embodiments, the masking agent can have pharmacological activity of its own after its release by cleavage of the tumor-associated protease cleavage site. The Cys substitution sites are selected such that replacing the original amino acid with a Cys does not detrimentally affect the ability of the antibody to specifically and strongly bind to its antigen.

Further, removal of the masking agent can leave behind a residual chemical group still covalently attached to the Cys.

In one embodiment, there is provided a prodrugged antibody according to formula (I):

(M−L)m−Ab I

wherein Ab is an antibody having at least one amino acid in its heavy or light chain variable region replaced by a Cys, wherein the replaced amino acid (a) is in a framework region; (b) has a side chain exposure of at least 30% and (c) is within 10 A, preferably 5 A, of a CDR amino acid; M is a masking agent that inhibits binding of Ab to its antigen; each L is, independently, a linker moiety bonded to M and Ab, L comprising a tumor-associated cleavage site and being bonded to Ab at aforesaid Cys; and m is 1, 2, 3, or 4.

In some embodiments, the at least one replaced amino in antibody Ab is at Kabat position 1, 3, 5, 19, 23, 25, 43, 46, 68, 72, 74, 75, 76, 82a, 82b, 83, 84, 85, or 105 of the heavy chain variable region or at Kabat position 1, 3, 5, 7, 8, 18, 20, 45, 57, 60, 63, 65, 66, 67, 69, 77, or 100 of the light chain variable region. In some embodiments, the at least one replaced amino acid in antibody Ab is at Kabat position 23 of the heavy chain or Kabat position 67 of the light chain. In some embodiments, there is provided an antibody having a Cys at Kabat position 67 of the light chain. The antibody can be an anti-CTLA4 antibody or an anti-CD137 antibody. In some embodiments, there is provided an antibody having a Cys at Kabat position 23 of the heavy chain. The antibody can be an anti-CTLA4 antibody or an anti-CD137 antibody.

The masked therapeutic antibody of the disclosure can be polyclonal, monoclonal, mouse, human, humanized, or chimeric. Suitable amino acids in the heavy and light chain variable regions for substitution with a Cys are framework amino acids whose side chains are solvent exposed—preferably at least 30% exposed—so that the substituted-in Cys is accessible for attachment of the masking agent. It is also important that the substituted-out amino acid is near a CDR amino acid, so that the masking agent can effectively interfere with antibody-antigen binding. A distance of no more than 10 A is preferred, more preferably no more than 5 A. Preferred positions for Cys substitution include positions 23 in the heavy chain variable region and 67 in the light chain variable region, numbering per Kabat. Both positions are in the framework region of the respective variable regions. A Cys can be substituted into these positions by site-specific substitution techniques well known in the art. A substitution at the first site can be referred to, using a shorthand notation, as VL X67C, where X denotes the substituted-out amino acid. In native antibodies, this site is highly conserved and is often Ser. A substitution at the second site can be similarly referred to as VH X23C.

A masked antibody of this disclosure can have either a substitution in the VH region or in the VL region, or both. If the antibody has only one of these substitutions, the theoretical maximum number of blocking moiety-linker compounds that can be attached is two, although a masked antibody preparation may assay statistically for a lower number, reflecting chemical inefficiency in the attachment process. If the antibody has both substitutions, the theoretical maximum number is four.

In some embodiments, the antibody is a bispecific antibody, which has two different pairs of heavy and light chains. Thus, a masked antibody of this disclosure can be a bispecific antibody in which only one heavy/light chain pair has been masked or one in which both heavy/light chain pairs have been masked. The substitution of an amino acid in a VH or VL region with a Cys, for the purpose of introducing a sulfhydryl side chain amenable to conjugation by maleimide addition chemistry to make an antibody-drug conjugate, is also known. See, for example, Eigenbrot et al. 2007 and Bhakta et a/. 2016).

Masking agents that can be used to interfere with or block activity of a masked antibody with its antigen include: polyethylene glycol (PEG), an albumin binding polypeptide, adnectin, a peptide, and a soluble globular protein such as albumin or fibrinogen. In some embodiments, the blocking agent comprises PEG having a molecular weight of at least about 2 kDa, with 2 kDa corresponding to PEG with about 45 —(CH2CH2O)— repeating units, and preferably PEG with a molecular weight of at least about 5 kDa, with 5 kDa corresponding to PEG with about 115 —(CH2CH2O)— repeating units.

An antibody having a Cys as described herein can be conjugated to a masking agent having a maleimide terminal group by Michael addition of the Cys sulfhydryl (SH), as known in the art. The procedures for such conjugation are well known in the art; see, for example, Shepard et al, WO 2017/112624 A 1 (2017), which is herein incorporated by reference. Further examples of specific masking agents for therapeutic antibodies are disclose in WO2019036433, which is herein incorporated by reference.

In further embodiments, the therapeutic agent may be an antibody in which variable regions are masked by linkage of the N-termini of variable regions chains to coiled-coil forming peptides. The coiled-coil forming peptides associate with one another to form coiled coils (i.e. , the respective peptides each form coils and these coils are coiled around each other). The coiled coils may sterically inhibit binding of the antibody binding site to its target. In some embodiments, the antibody comprises a bivalent antibody. Non-covalent associations between the coiled coil forming peptides are sufficient to form a stable coiled coils inhibiting binding of the antibody variable region; it is not for example necessary for the coiled-coil forming peptides to be further linked by a disulfide bridge between terminal cysteines of the respective peptides. The presence of non-naturally occurring cysteines is potentially disadvantageous because they can lead to misfolding or misconjugation problems. Masking of antibodies by this format can reduce binding affinities (and cytotoxic activities in the case of ADC's) by over a hundred fold. Antibodies can be masked in this format without significant impairment of expression, purification, conjugation, pharmacokinetics, or binding or other activity on unmasking.

In some embodiments, the masking agent comprises a coiled coil. Coiled coil forming peptides are peptide pairs that can associate with one another to form coiled coils. “Coiled coils” is a term of art referring to bundles of alpha-helices wound into superhelical structures. Leucine zipper forming peptides are one example of peptides associating to form coiled coils. The coiled coils formed in the current disclosure typically are formed from two coiled coil forming peptides. Coiled coils can form with alpha helices on the peptides in parallel or opposite orientations. Coiled coils are further characterized by packing of amino acid side chains in the core of the bundle, called knobs-into-holes, in which a residue from one helix (knob) packs into a space surrounded by four side chains of the facing helix (hole). The residues engaged in knobs-into-holes interactions are usually hydrophobic, whereas the outer residues are hydrophilic, the sequence of coiled coils therefore shows a ‘heptad’ repeat in the chemical nature of side chains. Examples of consensus formulae for heptad repeats in coiled coils forming peptides are provided by WO2011034605, which is herein incorporated by reference.

In some embodiments, the coiled coil comprises Formula II:

(XI, X2, X3, X4, X5, X6, X7)n II

wherein XI is a hydrophobic amino acid or asparagine; X2, X3 and X6 are any amino acid; X4 is a hydrophobic amino acid; and X5 and X7 are each a charged amino acid residue.

Examples of coiled coil comprise:

(SEQ ID NO: 58)
GASTSVDELQAEVDQLQDENYALKTKVAQLRKKVEKLSE;
(SEQ ID NO: 59)
GASTTVAQLRERVKTLRAQNYELESEVQRLREQVAQLA;
(SEQ ID NO: 60)
EACGASTSVDELQAEVDQLQDENYALKTKVAQLRKKVEKLSE;
(SEQ ID NO: 61)
EACGASTTVAQLRERVKTLRAQNYELESEVQRLREQVAQLA;
(SEQ ID NO: 62)
LEIEAAFLERENTALETRVAELRQRVQRARNRVSQYRTRY;
(SEQ ID NO: 63)
LEIRAAFLRQRNTALRTEVAELEQEVQRLENEVSQYETRY;
(SEQ ID NO: 64)
EACGALEIEAAFLERENTALETRVAELRQRVQRARNRVSQYRTRY;
(SEQ ID NO: 65)
EACGALEIRAAFLRQRNTALRTEVAELEQEVQRLENEVSQYETRY;
(SEQ ID NO: 66)
LEIRAAFLRRRNTALRTRVAELRQRVQRLRNIVSQYETRY;
(SEQ ID NO: 67)
LEIEAAFLEQENTALETEVAELEQEVQRLENIVSQYETRY;
(SEQ ID NO: 68)
EACGALEIRAAFLRRRNTALRTRVAELRQRVQRLRNIVSQYETRY;
(SEQ ID NO: 69)
EACGALEIEAAFLEQENTALETEVAELEQEVQRLENIVSQYETRY;
(SEQ ID NO: 70)
QGASTSVDELQAEVDQLEDENYALKTKVAQLRKKVEKL;
(SEQ ID NO: 71)
QGASTTVAQLEEKVKTLRAENYELKSEVQRLEEQVAQL;
(SEQ ID NO: 72)
EACGASTSVDELQAEVDQLEDENYALKTKVAQLRKKVEKL;
(SEQ ID NO: 73)
EACGASTTVAQLEEKVKTLRAENYELKSEVQRLEEQVAQL;
(SEQ ID NO: 74)
AGLTDTLQAETDQLEDKKSALQTEIANLLKEKEKLEFILAAH;
(SEQ ID NO: 75)
AGRIARLEEKVKTLKAQNSELASTANMLREQVAQLKQKVMNY;
(SEQ ID NO: 76)
EACGAGLTDTLQAETDQLEDKKSALQTEIANLLKEKEKLEFILAAH;
(SEQ ID NO: 77)
EACGAGRIARLEEKVKTLKAQNSELASTANMLREQVAQLKQKVMNY;
(SEQ ID NO: 78)
GKIAALKQKIAALKYKNAALKKKIAALKQ;
(SEQ ID NO: 79)
GEIAALEQEIAALEKENAALEWEIAALEQ;
(SEQ ID NO: 80)
EACGAGKIAALKQKIAALKYKNAALKKKIAALKQ;
and
(SEQ ID NO: 81)
EACGAGEIAALEQEIAALEKENAALEWEIAALEQ.

Coiled coils forming peptides are linked to the N-termini of antibody variable regions via a linker including a tumor-associated protease cleavage site. A typical antibody includes a heavy and light chain variable region, in which case a coiled-coil forming peptide is linked to the N-termini of each. A bivalent antibody has two binding sites, which may or may not be the same. In a normal monospecific antibody, the binding sites are the same and the antibody has two identical light and heavy chain pairs. In this case, each heavy chain is linked to the same coiled-coil forming peptide and each light chain to the same coiled-coil forming peptide (which may or may not be the same as the peptide linked to the heavy chain).

In a bispecific antibody, the binding sites are different and formed from two different heavy and light chain pairs. The binding sites can have specificity for different targets or different epitopes on the same target. If the binding sites have specificity for different targets, the targets can be on the same cell (e.g., two different surface antigens on a cancer cell) or two different cells (e.g., one surface antigen on a cancer cell and one on an immune cell such as a

T-cell). For example, one binding site of a bispecific antibody can be directed against CD3 or 4-1BB.

In a bispecific antibody, the heavy and light chain variable region of one binding site can be respectively linked to coiled-coil forming peptides. The heavy and light chain variable regions of the other binding site may or may not be also linked to coiled coil peptides. If the heavy light pairs of both binding sites are both linked to coiled coil peptides, then typically both heavy chain variable regions are linked to the same type of coiled-coil forming peptide as are both light chain variable regions. Masking of both binding sites can be useful, for example, if both binding sites have specificity for surface antigens on the same tumor. Masking of one but not both binding sites can be useful for example, when one binding site is specific for a tumor surface antigen and the other has specificity for a surface antigen on an immune cell. Either the binding site with specificity for the tumor surface antigen or for the immune cell antigen can be masked. Some bispecific antibodies with specificities to both a tumor surface antigen and an immune cell have masking of both sites.

Coiled coils can be formed from the same peptide forming a homodimer or two different peptides forming a heterodimer. For formation of a homodimer, light and heavy antibody chains are linked to the same coiled coil forming peptide. For formation of a heterodimer, light and heavy antibody chains are linked to different coiled coils peptides. For some pairs of coiled coil forming peptides, it is preferred that one of the pair be linked to the heavy chain and the other to the light chain of an antibody although the reverse orientation is also possible.

Each antibody chain can be linked to a single coiled coil forming peptide or multiple such peptides in tandem (e.g., two, three, four or five copies of a peptide). If the latter, the peptides in tandem linkage are usually the same. Also if tandem linkage is employed, light and heavy chains are usually linked to the same number of peptides.

Linkage of antibody chains to coiled coil forming peptides can reduce the binding affinity of an antibody by, for example, at least 10, 50, 100, 200, 500, 1000, 1500, 2000, 4000, 5000 or 10,000-fold relative to the same antibody without such linkage or after cleavage of such linkage. In some such antibodies, binding affinity is reduced 50-10,000, 50-5000, 50-4000, 50-1000, 100-10,000, 100-5000, 100-4000, 200-10,000, 200-5000, 50-1500, 100-1500, 200-1500, 200-1000, 500-1500, 50-1000, 100-1000, 200-1000, 500-1000, 50-500, 100-500 fold.

Antibodies include non-human, humanized, human, chimeric, and veneered antibodies, nanobodies, dAbs, scFV's, Fabs, and the like. Some such antibodies include immuno specific for a cancer cell antigen, preferably one on the cell surface internalizable within a cell on antibody binding. Targets to which antibodies can be directed include receptors on cancer cells and their ligands or counter-receptors (e.g., CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, and SLTRK-6).

In some embodiments, the antibody comprises brentuximab or brentuximab vedotin, anti-CD30, alemtuzumab, anti-CD52, rituximab, anti-CD20, trastuzumab Her/neu, nimotuzumab, cetuximab, anti-EGFR, bevacizumab, anti-VEGF, palivizumab, anti-RSV, abciximab, GpIIb/IIIa, infliximab, adalimumab, certolizumab, golimumab TNF-alpha, baciliximab, daclizumab, anti-IL-2, omalizumab, anti-IgE, gemtuzumab or vadastuximab, anti-CD33, natalizumab, anti-VLA-4, vedolizumab alpha4beta7, belimumab, anti-BAFF, otelixizumab, teplizumab, anti-CD3, ofatumumab, ocrelizumab, epratuzumab, anti-CD22, alemtuzumumab, eculizumab, canakimumab, mepolizumab, reslizumab, tocilizumab, ustekinumab, and briakinumab.

Further embodiments are described in WO2018107125, which is herein incorporated by reference.

### B. Collagen Binding Domain

Collagen is an extracellular matrix (ECM)-protein that regulates a variety of cellular biological functions, such as proliferation, differentiation, and adhesion in both normal and tumor tissue (Ricard-Blum, Cold Spring Harb Perspect Biol 3:a004978, 2011). Collagen is the most abundant protein in the mammalian body and exists in almost all tissues in one or more of 28 isoforms (Ricard-Blum, Cold Spring Harb Perspect Biol 3:a004978, 2011). The blood vessel sub-endothelial space is rich in collagen. Because of its insolubility under physiological conditions, collagen barely exists within the blood (Dubois et al., Blood 107:3902-06, 2006; Bergmeier and Hynes, Cold Spring Harb Perspect Biol 4:a005132, 2012). Tumor vasculature is reported to be permeable due to an abnormal structure (Nagy et al., British journal of cancer 100:865, 2009). Thus, with its leaky vasculature, collagen is exposed in the tumor (Liang et al., Journal of controlled release 209:101-109, 2015; Liang et al., Sci Rep 6:18205, 2016; Yasunaga et al., Bioconjugate chemistry 22:1776-83, 2011; Xu et al. The Journal of cell biology 154:1069-80, 2001; Swartz and Lund, Nat Rev Cancer 12:210-19). Also, tumor tissue contains increased amounts of collagen compared to normal tissues (Zhou et al. J Cancer 8:1466-76, 2017; Provenzano et al. BMC Med 6:11, 2008).

von Willebrand factor (vWF) is a blood coagulation factor and binds to both type I and type III collagen, and the adhesion receptor GPIb on blood platelets (Lenting et al., Journal of thrombosis and haemostasis:JTH 10:2428-37, 2012; Shahidi Advances in experimental medicine and biology 906:285-306, 2017). When injured, collagen beneath endothelial cells is exposed to blood plasma, and vWF-collagen binding initiates the thrombosis cascade (Shahidi Advances in experimental medicine and biology 906:285-306, 2017; Wu et al. Blood 99:3623-28, 2002). The vWF A domain has the highest affinity against collagen among reported non-bacterial origin proteins/peptides (Addi et al., Tissue Engineering Part B: Reviews, 2016). Particularly within the A domain, the A3 domain of vWF has been reported as a collagen binding domain (CBD) (Ribba et al. Thrombosis and haemostasis 86:848-54, 2001). As described above, the inventors contemplated that a fusion protein with the vWF A3 CBD may achieve targeted cytokine immunotherapy even when injected systemically due to exposure of collagen via the leaky tumor vasculature.

In some embodiments, the collagen binding domain comprises a polypeptide from decorin. Exemplary decorin polypeptides include human decorin, or a fragment thereof, which is represented by the following sequence:

CGPFQQRGLFDFMLEDEASGIGPEVPDDRDFEPSLGPVCPFRCQCHLRVVQC SDLGL DKVPKDLPPDTTLLDLQNNKITEIKDGDFKNLKNLHALILVNNKISKVSPGAFTPLVK LERLYL SKNQLKELPEKMPKTLQELRAHENEITKVRKVTFNGLNQMIVIELGTNPLKS SGIENGAFQGMKKLSYIRIADTNIT SIPQGLPP SLTELHLDGNKISRVDAASLKGLNNL AKLGLSFNSISAVDNGSLANTPHLRELHLDNNKLTRVPGGLAEHKYIQVVYLHNNNI

SVVGS SDFCPPGHNTKKASYSGVSLFSNPVQYWEIQPSTFRCVYVRSAIQLGNYK (SEQ ID NO:40), a peptide derived from human decorin: LRELHLDNNC (SEQ ID NO:41), and a peptide derived from bovine decorin: LRELHLNNNC (SEQ ID NO:44).

In some embodiments, the CBD comprises a polypeptide fragment from vWF. In some embodiments, the CBD comprises vWF Al derived from human sequence, residues 1237-1458 (474-695 of mature VWF) or a fragment thereof, which is represented by the amino acid sequence:

(SEQ ID NO: 39)
CQEPGGLVVPPTDAPVSPTTLYVEDISEPPLHDFYCSRLLDLVFLLDGSS
RLSEAEFEVLKAFVVDMMERLRISQKWVRVAVVEYHDGSHAYIGLKDRKR
PSELRRIASQVKYAGSQVASTSEVLKYTLFQIFSKIDRPEASRITLLLMA
SQEPQRIVISRNEVRYVQGLKKKKVIVIPVGIGPHANLKQIRLIEKQAPE
NKAFVLSSVDELEQQRDEIVSYLC.

In some embodiments, the CBP comprises all or a fragment of vWF A3, which is represented by the following amino acid sequences:

(SEQ ID NO: 37)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRICTG
and
(SEQ ID NO: 45)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRI.

In some embodiments, the CBP comprises vWF A3 domain polypeptide with a 6H tag with the following amino acid sequence:

(SEQ ID NO: 1)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRICTGHHHH
HH.

In some embodiments, the CBP comprises a peptide or polypeptide from von

Willebrand factor (vWF), such as a collagen binding peptide from vWF. The sequence of human vWF comprises the following:

(SEQ ID NO: 38)
MIPARFAGVLLALALILPGTLCAEGTRGRSSTARCSLFGSDFVNTFDGSM
YSFAGYCSYLLAGGCQKRSFSIIGDFQNGKRVSLSVYLGEFFDIHLFVNG
TVTQGDQRVSMPYASKGLYLETEAGYYKLSGEAYGFVARIDGSGNFQVLL
SDRYFNKTCGLCGNFNIFAEDDFMTQEGTLTSDPYDFANSWALSSGEQWC
ERASPPSSSCNISSGEMQKGLWEQCQLLKSTSVFARCHPLVDPEPFVALC
EKTLCECAGGLECACPALLEYARTCAQEGMVLYGWTDHSACSPVCPAGME
YRQCVSPCARTCQSLHINEMCQERCVDGCSCPEGQLLDEGLCVESTECPC
VHSGKRYPPGTSLSRDCNTCICRNSQWICSNEECPGECLVTGQSHFKSFD
NRYFTFSGICQYLLARDCQDHSFSIVIETVQCADDRDAVCTRSVTVRLPG
LHNSLVKLKHGAGVAMDGQDVQLPLLKGDLRIQHTVTASVRLSYGEDLQM
DWDGRGRLLVKLSPVYAGKTCGLCGNYNGNQGDDFLTPSGLAEPRVEDFG
NAWKLHGDCQDLQKQHSDPCALNPRMTRFSEEACAVLTSPTFEACHRAVS
PLPYLRNCRYDVCSCSDGRECLCGALASYAAACAGRGVRVAWREPGRCEL
NCPKGQVYLQCGTPCNLTCRSLSYPDEECNEACLEGCFCPPGLYMDERGD
CVPKAQCPCYYDGEIFQPEDIFSDHHTMCYCEDGFMHCTMSGVPGSLLPD
AVLSSPLSHRSKRSLSCRPPMVKLVCPADNLRAEGLECTKTCQNYDLECM
SMGCVSGCLCPPGMVRHENRCVALERCPCFHQGKEYAPGETVKIGCNTCV
CRDRKWNCTDHVCDATCSTIGMAHYLTFDGLKYLFPGECQYVLVQDYCGS
NPGTFRILVGNKGCSHPSVKCKKRVTILVEGGEIELFDGEVNVKRPMKDE
THFEVVESGRYIILLLGKALSVVWDRHLSISVVLKQTYQEKVCGLCGNFD
GIQNNDLTSSNLQVEEDPVDFGNSWKVSSQCADTRKVPLDSSPATCHNNI
MKQTMVDSSCRILTSDVFQDCNKLVDPEPYLDVCIYDTCSCESIGDCACF
CDTIAAYAHVCAQHGKVVTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAVVEYHDGSHAYIGLKDRKRPSELRRIASQVKYA
GSQVASTSEVLKYTLFQIFSKIDRPEASRITLLLMASQEPQRMSRNFVRY
VQGLKKKKVIVIPVGIGPHANLKQIRLIEKQAPENKAFVLSSVDELEQQR
DEIVSYLCDLAPEAPPPTLPPDMAQVTVGPGLLGVSTLGPKRNSMVLDVA
FVLEGSDKIGEADFNRSKEFMEEVIQRMDVGQDSIHVTVLQYSYMVTVEY
PFSEAQSKGDILQRVREIRYQGGNRTNTGLALRYLSDHSFLVSQGDREQA
PNLVYMVTGNPASDEIKRLPGDIQVVPIGVGPNANVQELERIGWPNAPIL
IQDFETLPREAPDLVLQRCCSGEGLQIPTLSPAPDCSQPLDVILLLDGSS
SFPASYFDEMKSFAKAFISKANIGPRLTQVSVLQYGSITTIDVPWNVVPE
KAHLLSLVDVMQREGGPSQIGDALGFAVRYLTSEMHGARPGASKAVVILV
TDVSVDSVDAAADAARSNRVTVFPIGIGDRYDAAQLRILAGPAGDSNVVK
LQRIEDLPTMVTLGNSFLHKLCSGFVRICMDEDGNEKRPGDVWTLPDQCH
TVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEV
RFNHLGHIFTFTPQNNEFQLQLSPKTFASKTYGLCGICDENGANDFMLRD
GTVTTDWKTLVQEWTVQRPGQTCQPILEEQCLVPDSSHCQVLLLPLFAEC
HKVLAPATFYAICQQDSCHQEQVCEVIASYAHLCRTNGVCVDWRTPDFCA
MSCPPSLVYNHCEHGCPRHCDGNVSSCGDHPSEGCFCPPDKVMLEGSCVP
EEACTQCIGEDGVQHQFLEAWVPDHQPCQICTCLSGRKVNCTTQPCPTAK
APTCGLCEVARLRQNADQCCPEYECVCDPVSCDLPPVPHCERGLQPTLTN
PGECRPNFTCACRKEECKRVSPPSCPPHRLPTLRKTQCCDEYECACNCVN
STVSCPLGYLASTATNDCGCTTTTCLPDKVCVHRSTIYPVGQFWEEGCDV
CTCTDMEDAVMGLRVAQCSQKPCEDSCRSGFTYVLHEGECCGRCLPSACE
VVTGSPRGDSQSSWKSVGSQWASPENPCLINECVRVKEEVFIQQRNVSCP
QLEVPVCPSGFQLSCKTSACCPSCRCERMEACMLNGTVIGPGKTVMIDVC
TTCRCMVQVGVISGFKLECRKTTCNPCPLGYKEENNTGECCGRCLPTACT
IQLRGGQINITLKRDETLQDGCDTHFCKVNERGEYFWEKRVTGCPPFDEH
KCLAEGGKIMKIPGTCCDTCEEPECNDITARLQYVKVGSCKSEVEVDIHY
CQGKCASKAMYSIDINDVQDQCSCCSPTRTEPMQVALHCTNGSVVYHEVL
NAMECKCSPRKCSK.

In some embodiments, the peptide is from the vWF A3 domain and has the following amino acid sequence (or a fragment thereof):

(SEQ ID NO: 46)
CSGEGLQIPTLSPAPDCSQPLDVILLLDGSSSFPASYFDEMKSFAKAFIS
KANIGPRLTQVSVLQYGSITTIDVPWNVVPEKAHLLSLVDVMQREGGPSQ
IGDALGFAVRYLTSEMHGARPGASKAVVILVTDVSVDSVDAAADAARSNR
VTVFPIGIGDRYDAAQLRILAGPAGDSNVVKLQRIEDLPTMVTLGNSFLH
KLCSG.

The CBP peptide or polypeptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a CBD peptide or fragment of the peptides described above, such as SEQ ID NOS:1, 37-41, and 44-46.

### C. Linker

In some embodiments, the polypeptides comprise or further comprise a linker. The linker may be between any two domains of the polypeptide. In some embodiments, the polypeptide comprises a linker between the CBP and the cytokine. In some embodiments, the polypeptide comprises a linker in between the CBP and the serum polypeptide. In some embodiments, the polypeptide comprises a linker in between the masking agent and the cytokine. In some embodiments, the polypeptide comprises a linker in between the albumin and the cytokine. In some embodiments, the polypeptide comprises a linker in between the therapeutic agent and the masking agent. In some embodiments, the polypeptide comprises a linker in between the therapeutic agent and the CBP. In some embodiments, the linker comprises one or more tumor-associated protease cleavage sites. A tumor-associated protease cleavage site refers to a cleavage site that is recognized by a protease that is highly upregulated or enriched in the tumor microenvironment. While the tumor-associated protease cleavage site may not be tumor-specific, meaning that the protease is only expressed in the tumor, it is tumor-enriched, meaning that the protease is expressed at a level in the tumor microenvironment that is higher than normal tissues or most normal tissues. In some embodiments, the tumor-associated protease cleavage site comprises an amino acid sequence that is recognized and cleaved by a matrix metalloproteinase. For example, the tumor-associated protease cleavage site may be one that is cleaved by MMP1, MMP2, MMP3, MMP7, MMP8, MMP9, MMP10, MMP11, MMP12, MMP13, MMP14, MMP15, MMP16, MMP17, MMP18, MMP19, MNIP20, MMP21, MNIP23A, MNIP23B, MMP24, MMP25, MMP26, MMP27, MMP28, or combinations thereof. In some embodiments, the tumor-associated protease cleavage site comprises the MMP-response sequence of SEQ ID NO:13: GLLSGRSDNH. In some embodiments, the tumor-associated protease cleavage site may be one that is cleaved by thrombin. In some embodiments, the thrombin-responsive sequence comprises SEQ ID NO:14: LVPRGS.

In some embodiments, two polypeptides such as a two of a CBP, serum protein, therapeutic agent, masking agent, and cytokine may be linked through a bifunctional linker. Linkers, such as amino acid or peptidimimetic sequences may be inserted between the peptide and/or antibody sequence. In an embodiment, a fynomer domain is joined to a Heavy (H) chain or Light (L) chain immediately after the last amino acid at the amino(NH2)-terminus or the carboxy(C)-terminus of the Heavy (H) chain or the Light (L) chain. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. For example, a suitable peptide linker may be GGGSGGGS (SEQ ID NO:47) or (GGGS)n (SEQ ID NO:48), wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as CN (where N=1-100 carbon atoms, e.g. N=1, 2, 3, 4, 5, 6, 7, 8, 9, 10, 11, 12, 13, 14, or 15, or more). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, -SH), while its sulfo-NHS ester is reactive toward primary amines (as found in lysine and the protein or peptide N-terminus). Further, the linker may be maleimidocaproyl (mc).

In some embodiments, the linker comprises one or more polypeptides that are cleavable by—i.e., are a substrate for—an enzyme (a protease) that is uniquely expressed or overexpressed in a cancer or tumor microenvironment, compared to healthy tissue or organ. Preferably, the enzyme is found in the extracellular environment of a tumor. Examples of such proteases include: aspartate proteases (e.g., renin), fibroblast activation protein (FAP), aspartic cathepsins (e.g., cathepsin D, caspase 1, caspase 2, etc.), cysteine cathepsins (e.g., cathepsin B), cysteine proteases (e.g., legumain), disintegrin/metalloproteinases (ADAMs, e.g., ADAMS, ADAMS), disintegrin/metalloproteinases with thrombospondin motifs (ADAMTS, e.g., ADAMTS1), integral membrane serine proteases (e.g., matriptase 2, MT-SP1/matriptase, TMPRSS2, TMPRSS3, TMPRSS4), kallikrein-related peptidases (KLKs, e.g. KLK4, KLK5), matrix metalloproteases (e.g., MMP-1, MMP-2, MMP-9), and serine proteases (e.g., cathepsin A, coagulation factor proteases such as elastase, plasmin, thrombin, PSA, uPA, Factor Vila, Factor Xa, and HCV NS3/4). Preferably, the protease is fibroblast activation protein (FAP), urokinase-type plasminogen activator (uPA, urokinase), MT-SP1/matriptase, legumain, or a matrix metalloprotease (especially MMP-1, MMP-2, and MMP-9). Those skilled in the art will appreciate that the choice of the enzyme and the corresponding cleavable peptide will depend on the disease to be treated and the protease(s) expressed by the affected tissue or organ.

Further examples of tumor-associated protease sites include LSGRSDNH (SEQ ID NO:49), cleaved by urokinase, matriptase, or legumain; VPLSLYS (SEQ ID NO:50), cleaved by MMP2 or MMP9; PLGLAG (SEQ ID NO:51), cleaved by MMP2; VLVPMAMMAS (SEQ

ID NO:52), cleaved by MMPI; XXQAR(A/V)X (SEQ ID NO:53), where Xis any amino acid, cleaved by Matriptase; AGPR (SEQ ID NO:54), cleaved by matriptase; AANL (SEQ ID NO:55) and PTNL (SEQ ID NO:56), cleaved by Legumain; and TSGRSANP (SEQ ID NO:57).

A linker sequence may be included in the polypeptides of the disclosures. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate or be between any two of a CBP, serum protein, therapeutic agent, masking agent, and cytokine.

The linker may comprise a sequence of SEQ ID NO:13, 14, or 47-57 or a peptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:13, 14, or 47-57.

Further examples of tumor-associated protease sites include those in the table below:

| Substrate | SEQ ID | | Substrate | SEQ ID | |
|---|---|---|---|---|---|
| Sequence | NO | Protease | Sequence | NO | Protease |
| YLGRSYKV | 109 | C1s | RPLALWRS | 152 | MMP7 |
| FK | | Cathepsin B | NKSRLGLG | 153 | MMP7/ |
| | | | | | Cathpsin B |
| TSQVNGLN | 110 | Elastase2 | GPQGIAGQR | 154 | MMP9 |
| APPPVVLL | 111 | Matrilysin | PVGLIG | 155 | MMP2/9 |
| | | (MMP7) | | | |
| GGGGF | 112 | Cathepsin B | KPVSLSYR | 156 | MMP9 |
| TPEHVVPY | 113 | Endothelin- | QPVGINTS | 157 | MMP3 |
| | | converting | | | (stromelysin-1) |
| | | enzyme 1 | | | |
| PRSFFRLGK | 114 | Cathepsin D | PLGMTS | 158 | MMP9 |
| PQGRIVGG | 115 | Hepsin | PRALM | 159 | MMP9 |
| EVLLSWAV | 116 | Cathepsin G | GPLPLR | 160 | MT1-MMP |
| AANL | 55 | Legumain | KQLRVVNG | 161 | MT-SP1/ST14/ |
| | | | | | uPA/Hepsin |
| PVSLSYRC | 117 | Cathepsin G | PLGLYA | 162 | Pan-MMP |
| LRELHLDN | 118 | Matrilysin | AFK | | Plasmin |
| | | (MMP7) | | | |
| AAPV | 119 | Elastase 2 | LVPRGS | 14 | Thrombin |
| IPENFFGV | 120 | Matrilysin | GGR | | Plasmin/ |
| | | (MMP7) | | | TMPRSS2 |
| PRFKIIGG | 121 | Hepsin | HSSKLQL | 163 | PSA |
| MLEDEASG | 122 | Matrilysin | SSKYQ | 164 | PSA |
| | | (MMP7) | | | |
| KQSRKFVP | 123 | Matriptase | GPLGVRGK | 165 | MMP2 |
| | | (ST14) | | | |
| RQARVVGG | 124 | Matriptase2/ | GGGSGGGS | 47 | Control |
| | | Hepsin | | | |
| GKAFRRL | 125 | Hk2 | PRGMAS | 166 | MMP9 |
| GPLGLWAQ | 126 | MMP (esp2/9) | AAALGNVAP | 167 | MMP9 |
| SLGRKIQI | 127 | MASP2 | GGSGRSANA | 168 | uPA |
| PVSLR | 128 | MMP1 | PLGVRG | 169 | MMP2/9 |
| AAATSIAM | 129 | MMP11 | SGRSANAK | 170 | uPA |
| | | (stromelysin-3) | | | |
| AAGAMFLE | 130 | MMP11 | GPLGLWAGG | 171 | MMP2 |
| | | (stromelysin-3) | | | |
| EAAAATSI | 131 | MMP11 | SGRSA | 172 | uPA |
| | | (stromelysin-3) | | | |
| PRHLR | 132 | MMP14 | PLGLYL | 173 | MMP2/9 |
| PRGLRK | 133 | MMP14 | SRRRVNSL | 174 | uPA |
| VPLSLYSG | 134 | MMP (esp 2/9) | GSTFF | 175 | Cathepsin D |
| PRGLRP | 135 | MMP15/16/24/25 | CPGRVVGG | 176 | uPa/tPA |
| PRHLRN | 136 | MMP15/16/24/25 | GTQFF | 177 | Cathepsin D |
| PRWLRS | 137 | MMP15/16/24/25 | PQGLAG | 178 | MMP9 |
| HPVGLLAR | 138 | MMP2 | QVVAG | 179 | Cathepsin B |
| KGPLGVRG | 139 | MMP2 | TYSRSRYL | 180 | uPA |
| CGLDD | 140 | MMP2/9 | NSGRAVTY | 181 | uPA |
| STAVIVSA | 141 | MMP3 | GPQGARGQ | 182 | MMP9 |
| | | (stromelysin-1) | | | |
| PLGLAG | 51 | MMP2 | PSSRRRVN | 183 | uPA |
| GPLGIAGQ | 142 | MMP2/9 | PLGLYAL | 184 | MMP2/9 |
| PLGLWA | 143 | MMP2 | PMKRLTLG | 185 | Cathepsin B |
| GPLGMLSQ | 144 | MMP2/9 | DDDKIVGG | 186 | Cathepsin B |
| PLGVRGK | 145 | MMP2 | HLVEALYL | 187 | Cathepsin B |
| GPQGIWGQ | 146 | MMP2/9, | EVDLLIGS | 188 | Cathepsin B |
| | | MT1-MMP | | | |
| AIPVSLR | 147 | MMP2 | PRFKIIGG | 121 | Cathepsin B |
| GPLGLARK | 148 | MMP7 | AVRWLLTA | 189 | MMP9 |
| PQGIAMG | 149 | MMP2 | LSGRSDNH | 49 | uPA/ |
| | | | | | matriptase/ |
| | | | | | legumain |
| VASSSTAV | 150 | MMP3 | GGGRR | 190 | uPA |
| | | (stromelysin-1) | | | |
| PLGL | 151 | MMP2/9 | | | |

### D. Serum Proteins

In some embodiments, the polypeptides of the disclosure are further linked to a serum protein. Serum proteins include, for example, albumin, globulin, and fibrinogen. Globulins include alpha 1 globulins, alpha 2 globulins, beta globulins, and gamma globulins.

The albumin may be mouse, human, bovine, or any other homologous albumin protein. In some embodiments, the albumin comprises human serum albumin, which is encoded by the ALB gene, and exemplified by the following amino acid sequence:

(SEQ ID NO: 43)
KWVTFISLLFLFSSAYSRGVERRDAHKSEVAHREKDLGEENFKALVLIAF
AQYLQQCPFEDHVKLVNEVTEFAKTCVADESAENCDKSLHTLFGDKLCTV
ATLRETYGEMADCCAKQEPERNECFLQHKDDNPNLPRLVRPEVDVMCTAF
HDNEETFLKKYLYEIARRHPYFYAPELLFFAKRYKAAFTECCQAADKAAC
LLPKLDELRDEGKASSAKQRLKCASLQKFGERAFKAWAVARLSQRFPKAE
FAEVSKLVTDLTKVHTECCHGDLLECADDRADLAKYICENQDSISSKLKE
CCEKPLLEKSHCIAEVENDEMPADLPSLAADFVESKDVCKNYAEAKDVFL
GMFLYEYARRHPDYSVVLLLRLAKTYETTLEKCCAAADPHECYAKVEDEF
KPLVEEPQNLIKQNCELFEQLGEYKEQNALLVRYTKKVPQVSTPTLVEVS
RNLGKVGSKCCKHPEAKRMPCAEDYLSVVLNQLCVLHEKTPVSDRVTKCC
TESLVNRRPCFSALEVDETYVPKEFNAETFTFHADICTLSEKERQIKKQT
ALVELVKHKPKATKEQLKAVMDDFAAFVEKCCKADDKETCFAEEGKKLVA
ASQAALGL.

In some embodiments, the albumin comprises mouse albumin having the following sequence:

(SEQ ID NO: 42)
EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA.

In some embodiments, the serum protein comprises a polypeptide of SEQ ID NO:42 or 43, ora fragment thereof, or a polypeptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:42, 43, or a fragment thereof.

### E. Polypeptide Embodiments

Specific polypeptide embodiments are exemplified below:

| mIL12Rβ1- | QLGASGPGDGCCVEKTSFPEGASGSPLGPRNLSCYRVS |
|---|---|
| Thrombin- | KTDYECSWQYDGPEDNVSHVLWCCFVPPNHTHTGQERC |
| linker- | RYFSSGPDRTVQFWEQDGIPVLSKVNFWVESRLGNRTM |
| IL12p35 | KSQKISQYLYNWTKTTPPLGHIKVSQSHRQLRMDWNVS |
| | EEAGAEVQFRRRMPTTNWTLGDCGPQVNSGSGVLGDIR |
| | GSMSESCLCPSENMAQEIQIRRRRRLSSGAPGGPWSDW |
| | SMPVCVPPEVLPQALVPRGSGGGSGGGSRVIPVSGPAR |
| | CLSQSRNLLKTTDDMVKTAREKLKHYSCTAEDIDHEDI |
| | TRDQTSTLKTCLPLELHKNESCLATRETSSTTRGSCLP |
| | PQKTSLMMTLCLGSIYEDLKMYQTEFQAINAALQNHNH |
| | QQIILDKGMLVAIDELMQSLNHNGETLRQKPPVGEADP |
| | YRVKMKLCILLHAFSTRVVTINRVMGYLSSA (SEQ |
| | ID NO: 7) |
| CBD- | QLGASGPGDGCCVEKTSFPEGASGSPLGPRNLSCYRVS |
| mIL12Rβ1- | KTDYECSWQYDGPEDNVSHVLWCCFVPPNHTHTGQERC |
| Thrombin- | RYFSSGPDRTVQFWEQDGIPVLSKVNFWVESRLGNRTM |
| linker- | KSQKISQYLYNWTKTTPPLGHIKVSQSHRQLRMDWNVS |
| IL12p40 | EEAGAEVQFRRRMPTTNWTLGDCGPQVNSGSGVLGDIR |
| | GSMSESCLCPSENMAQEIQIRRRRRLSSGAPGGPWSDW |
| | SMPVCVPPEVLPQALVPRGSGGGSGGGSGGGSGGGSGG |
| | GSGGGSMWELEKDVYVVEVDWTPDAPGETVNLTCDTPE |
| | EDDITWTSDQRHGVIGSGKTLTITVKEFLDAGQYTCHK |
| | GGETLSHSHLLLHKKENGIWSTEILKNFKNKTFLKCEA |
| | PNYSGRFTCSWLVQRNMDLKFNIKSSSSSPDSRAVTCG |
| | MASLSAEKVTLDQRDYEKYSVSCQEDVTCPTAEETLPI |
| | ELALEARQQNKYENYSTSFFIRDIIKPDPPKNLQMKPL |
| | KNSQVEVSWEYPDSWSTPHSYFSLKFFVRIQRKKEKMK |
| | ETEEGCNQKGAFLVEKTSTEVQCKGGNVCVQAQDRYYN |
| | SSCSKWACVPCRVRS (SEQ ID NO: 8) |
| mIL12Rβ1- | QLGASGPGDGCCVEKTSFPEGASGSPLGPRNLSCYRVS |
| MMPlinker- | KTDYECSWQYDGPEDNVSHVLWCCFVPPNHTHTGQERC |
| IL12p35 | RYFSSGPDRTVQFWEQDGIPVLSKVNFWVESRLGNRTM |
| | KSQKISQYLYNWTKTTPPLGHIKVSQSHRQLRMDWNVS |
| | EEAGAEVQFRRRMPTTNWTLGDCGPQVNSGSGVLGDIR |
| | GSMSESCLCPSENMAQEIQIRRRRRLSSGAPGGPWSDW |
| | SMPVCVPPEVLPQAGLLSGRSDNHGGGSGGGSRVIPVS |
| | GPARCLSQSRNLLKTTDDMVKTAREKLKHYSCTAEDID |
| | HEDITRDQTSTLKTCLPLELHKNESCLATRETSSTTRG |
| | SCLPPQKTSLMMTLCLGSIYEDLKMYQTEFQAINAALQ |
| | NHNHQQIILDKGMLVAIDELMQSLNHNGETLRQKPPVG |
| | EADPYRVKMKLCILLHAFSTRVVTINRVMGYLSSAHHH |
| | HHH (SEQ ID NO: 9) |
| mIL12Rβ1- | CCVEKTSFPEGASGSPLGPRNLSCYRVSKTDYECSWQY |
| MMPlinker- | DGPEDNVSHVLWCCFVPPNHTHTGQERCRYFSSGPDRT |
| IL12p40 | VQFWEQDGIPVLSKVNFWVESRLGNRTMKSQKISQYLY |
| | NWTKTTPPLGHIKVSQSHRQLRMDWNVSEEAGAEVQFR |
| | RRMPTTNWTLGDCGPQVNSGSGVLGDIRGSMSESCLCP |
| | SENMAQEIQIRRRRRLSSGAPGGPWSDWSMPVCVPPEV |
| | LPQAGLLSGRSDNHGGGSGGGSGGGSGGGSGGGSGGGS |
| | MWELEKDVYVVEVDWTPDAPGETVNLTCDTPEEDDITW |
| | TSDQRHGVIGSGKTLTITVKEFLDAGQYTCHKGGETLS |
| | HSHLLLHKKENGIWSTEILKNFKNKTFLKCEAPNYSGR |
| | FTCSWLVQRNMDLKFNIKSSSSSPDSRAVTCGMASLSA |
| | EKVTLDQRDYEKYSVSCQEDVTCPTAEETLPIELALEA |
| | RQQNKYENYSTSFFIRDIIKPDPPKNLQMKPLKNSQVE |
| | VSWEYPDSWSTPHSYFSLKFFVRIQRKKEKMKETEEGC |
| | NQKGAFLVEKTSTEVQCKGGNVCVQAQDRYYNSSCSKW |
| | ACVPCRVRS (SEQ ID NO: 10) |
| CBD- | CSQPLDVILLLDGSSSFPASYFDEMKSFAKAFISKANI |
| mIL12Rβ1- | GPRLTQVSVLQYGSITTIDVPWNVVPEKAHLLSLVDVM |
| Thrombin- | QREGGPSQIGDALGFAVRYLTSEMHGARPGASKAVVIL |
| linker- | VTDVSVDSVDAAADAARSNRVTVFPIGIGDRYDAAQLR |
| IL12p40 | ILAGPAGDSNVVKLQRIEDLPTMVTLGNSFLHKLCSGF |
| | VRIGGGSGGGSQLGASGPGDGCCVEKTSFPEGASGSPL |
| | GPRNLSCYRVSKTDYECSWQYDGPEDNVSHVLWCCFVP |
| | PNHTHTGQERCRYFSSGPDRTVQFWEQDGIPVLSKVNF |
| | WVESRLGNRTMKSQKISQYLYNWTKTTPPLGHIKVSQS |
| | HRQLRMDWNVSEEAGAEVQFRRRMPTTNWTLGDCGPQV |
| | NSGSGVLGDIRGSMSESCLCPSENMAQEIQIRRRRRLS |
| | SGAPGGPWSDWSMPVCVPPEVLPQALVPRGSGGGSGGG |
| | SGGGSGGGSGGGSGGGSMWELEKDVYVVEVDWTPDAPG |
| | ETVNLTCDTPEEDDITWTSDQRHGVIGSGKTLTITVKE |
| | FLDAGQYTCHKGGETLSHSHLLLHKKENGIWSTEILKN |
| | FKNKTFLKCEAPNYSGRFTCSWLVQRNMDLKFNIKSSS |
| | SSPDSRAVTCGMASLSAEKVTLDQRDYEKYSVSCQEDV |
| | TCPTAEETLPIELALEARQQNKYENYSTSFFIRDIIKP |
| | DPPKNLQMKPLKNSQVEVSWEYPDSWSTPHSYFSLKFF |
| | VRIQRKKEKMKETEEGCNQKGAFLVEKTSTEVQCKGGN |
| | VCVQAQDRYYNSSCSKWACVPCRVRS (SEQ ID NO: |
| | 11) |
| CBD- | CSQPLDVILLLDGSSSFPASYFDEMKSFAKAFISKANI |
| mIL12Rβ1- | GPRLTQVSVLQYGSITTIDVPWNVVPEKAHLLSLVDVM |
| MMPlinker- | QREGGPSQIGDALGFAVRYLTSEMHGARPGASKAVVIL |
| IL12p40 | VTDVSVDSVDAAADAARSNRVTVFPIGIGDRYDAAQLR |
| | ILAGPAGDSNVVKLQRIEDLPTMVTLGNSFLHKLCSGF |
| | VRIGGGSGGGSQLGASGPGDGCCVEKTSFPEGASGSPL |
| | GPRNLSCYRVSKTDYECSWQYDGPEDNVSHVLWCCFVP |
| | PNHTHTGQERCRYFSSGPDRTVQFWEQDGIPVLSKVNF |
| | WVESRLGNRTMKSQKISQYLYNWTKTTPPLGHIKVSQS |
| | HRQLRMDWNVSEEAGAEVQFRRRMPTTNWTLGDCGPQV |
| | NSGSGVLGDIRGSMSESCLCPSENMAQEIQIRRRRRLS |
| | SGAPGGPWSDWSMPVCVPPEVLPQAGLLSGRSDNHGGG |
| | SGGGSGGGSGGGSGGGSGGGSMWELEKDVYVVEVDWTP |
| | DAPGETVNLTCDTPEEDDITWTSDQRHGVIGSGKTLTI |
| | TVKEFLDAGQYTCHKGGETLSHSHLLLHKKENGIWSTE |
| | ILKNFKNKTFLKCEAPNYSGRFTCSWLVQRNMDLKFNI |
| | KSSSSSPDSRAVTCGMASLSAEKVTLDQRDYEKYSVSC |
| | QEDVTCPTAEETLPIELALEARQQNKYENYSTSFFIRD |
| | IIKPDPPKNLQMKPLKNSQVEVSWEYPDSWSTPHSYFS |
| | LKFFVRIQRKKEKMKETEEGCNQKGAFLVEKTSTEVQC |
| | KGGNVCVQAQDRYYNSSCSKWACVPCRVRS (SEQ ID |
| | NO: 12) |
| mIL12Rβ1- | QLGASGPGDGCCVEKTSFPEGASGSPLGPRNLSCYRVS |
| Thrombin- | KTDYECSWQYDGPEDNVSHVLWCCFVPPNHTHTGQERC |
| MMP- | RYFSSGPDRTVQFWEQDGIPVLSKVNFWVESRLGNRTM |
| MMPlinker- | KSQKISQYLYNWTKTTPPLGHIKVSQSHRQLRMDWNVS |
| IL12p35 | EEAGAEVQFRRRMPTTNWTLGDCGPQVNSGSGVLGDIR |
| | GSMSESCLCPSENMAQEIQIRRRRRLSSGAPGGPWSDW |
| | SMPVCVPPEVLPQALVPRGSGLLSGRSDNHGLLSGRSD |
| | NHGGGSGGGSRVIPVSGPARCLSQSRNLLKTTDDMVKT |
| | AREKLKHYSCTAEDIDHEDITRDQTSTLKTCLPLELHK |
| | NESCLATRETSSTTRGSCLPPQKTSLMMTLCLGSIYED |
| | LKMYQTEFQAINAALQNHNHQQIILDKGMLVAIDELMQ |
| | SLNHNGETLRQKPPVGEADPYRVKMKLCILLHAFSTRV |
| | VTINRVMGYLSSA (SEQ ID NO: 15) |
| mIL12Rβ1- | CCVEKTSFPEGASGSPLGPRNLSCYRVSKTDYECSWQY |
| MPMMPMMP- | DGPEDNVSHVLWCCFVPPNHTHTGQERCRYFSSGPDRT |
| linker- | VQFWEQDGIPVLSKVNFWVESRLGNRTMKSQKISQYLY |
| IL12p35 | NWTKTTPPLGHIKVSQSHRQLRMDWNVSEEAGAEVQFR |
| | RRMPTTNWTLGDCGPQVNSGSGVLGDIRGSMSESCLCP |
| | SENMAQEIQIRRRRRLSSGAPGGPWSDWSMPVCVPPEV |
| | LPQAGLLSGRSDNHGLLSGRSDNHGLLSGRSDNHGGGS |
| | GGGSRVIPVSGPARCLSQSRNLLKTTDDMVKTAREKLK |
| | HYSCTAEDIDHEDITRDQTSTLKTCLPLELHKNESCLA |
| | TRETSSTTRGSCLPPQKTSLMMTLCLGSIYEDLKMYQT |
| | EFQAINAALQNHNHQQIILDKGMLVAIDELMQSLNHNG |
| | ETLRQKPPVGEADPYRVKMKLCILLHAFSTRVVTINRV |
| | MGYLSSA (SEQ ID NO: 16) |
| mIL12Rβ1- | CCVEKTSFPEGASGSPLGPRNLSCYRVSKTDYECSWQY |
| Thrombin- | DGPEDNVSHVLWCCFVPPNHTHTGQERCRYFSSGPDRT |
| Thrombin- | VQFWEQDGIPVLSKVNFWVESRLGNRTMKSQKISQYLY |
| Thrombin- | NWTKTTPPLGHIKVSQSHRQLRMDWNVSEEAGAEVQFR |
| linker- | RRMPTTNWTLGDCGPQVNSGSGVLGDIRGSMSESCLCP |
| IL12p35 | SENMAQEIQIRRRRRLSSGAPGGPWSDWSMPVCVPPEV |
| | LPQALVPRGSLVPRGSLVPRGSGGGSGGGSRVIPVSGP |
| | ARCLSQSRNLLKTTDDMVKTAREKLKHYSCTAEDIDHE |
| | DITRDQTSTLKTCLPLELHKNESCLATRETSSTTRGSC |
| | LPPQKTSLMMTLCLGSIYEDLKMYQTEFQAINAALQNH |
| | NHQQIILDKGMLVAIDELMQSLNHNGETLRQKPPVGEA |
| | DPYRVKMKLCILLHAFSTRVVTINRVMGYLSSA (SEQ |
| | ID NO: 17) |
| mIL12Rβ1- | CCVEKTSFPEGASGSPLGPRNLSCYRVSKTDYECSWQY |
| Throm3- | DGPEDNVSHVLWCCFVPPNHTHTGQERCRYFSSGPDRT |
| MMP3- | VQFWEQDGIPVLSKVNFWVESRLGNRTMKSQKISQYLY |
| linker- | NWTKTTPPLGHIKVSQSHRQLRMDWNVSEEAGAEVQFR |
| IL12p35 | RRMPTTNWTLGDCGPQVNSGSGVLGDIRGSMSESCLCP |
| | SENMAQEIQIRRRRRLSSGAPGGPWSDWSMPVCVPPEV |
| | LPQALVPRGSLVPRGSLVPRGSGLLSGRSDNHGLLSGR |
| | SDNHGLLSGRSDNHGGGSGGGSRVIPVSGPARCLSQSR |
| | NLLKTTDDMVKTAREKLKHYSCTAEDIDHEDITRDQTS |
| | TLKTCLPLELHKNESCLATRETSSTTRGSCLPPQKTSL |
| | MMTLCLGSIYEDLKMYQTEFQAINAALQNHNHQQIILD |
| | KGMLVAIDELMQSLNHNGETLRQKPPVGEADPYRVKMK |
| | LCILLHAFSTRVVTINRVMGYLSSA (SEQ ID NO: |
| | 18) |

In some embodiments, the polypeptide has at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of the disclosure, such as SEQ ID NOS:7-12, and 15-18, or fragments thereof.

### F. Protein Tags

In some embodiments, the polypeptide further comprises a protein tag. The protein tag can be used for protein purification and/or immunoassays, for example. Exemplary protein tags include AviTag, a peptide allowing biotinylation by the enzyme BirA and so the protein can be isolated by streptavidin (GLNDIFEAQKIEWHE (SEQ ID NO:82)), Calmodulin-tag, a peptide bound by the protein calmodulin (KRRWKKNFIAVSAANRFKKISSSGAL (SEQ ID NO:83)), polyglutamate tag, a peptide binding efficiently to anion-exchange resin such as Mono-Q (EEEEEE (SEQ ID NO:84)), E-tag, a peptide recognized by an antibody (GAPVPYPDPLEPR (SEQ ID NO:102)), FLAG-tag, a peptide recognized by an antibody (DYKDDDDK (SEQ ID NO:85)), HA-tag, a peptide from hemagglutinin recognized by an antibody (YPYDVPDYA (SEQ ID NO:86)), His-tag, 5-10 histidines bound by a nickel or cobalt chelate (HEIHEIHH (SEQ ID NO:87)), Myc-tag, a peptide derived from c-myc recognized by an antibody (EQKLISEEDL (SEQ ID NO:88)), NE-tag, a novel 18-amino-acid synthetic peptide (TKENPRSNQEESYDDNES (SEQ ID NO:89)) recognized by a monoclonal IgG1 antibody, which is useful in a wide spectrum of applications including Western blotting, ELISA, flow cytometry, immunocytochemistry, immunoprecipitation, and affinity purification of recombinant proteins, S-tag, a peptide derived from Ribonuclease A (KETAAAKFERQHMDS (SEQ ID NO:90)), SBP-tag, a peptide which binds to streptavidin (MDEKTTGWRGGHVVEGLAGELEQLRARLEHHPQGQREP (SEQ ID NO:91)), Softag 1, for mammalian expression (SLAELLNAGLGGS (SEQ ID NO:92)), Softag 3, for prokaryotic expression (TQDPSRVG (SEQ ID NO:93)), Strep-tag, a peptide which binds to streptavidin or the modified streptavidin called streptactin (Strep-tag II: WSHPQFEK (SEQ ID NO:94)), TC tag, a tetracysteine tag that is recognized by FlAsH and ReAsH biarsenical compounds (CCPGCC (SEQ ID NO:95)), V5 tag, a peptide recognized by an antibody (GKPIPNPLLGLDST (SEQ ID NO:96)), VSV-tag, a peptide recognized by an antibody (YTDIEMNRLGK (SEQ ID NO:97)), Xpress tag (DLYDDDDK (SEQ ID NO:98)), Covalent peptide tags, Isopeptag, a peptide which binds covalently to pilin-C protein (TDKDMTITFTNKKDAE (SEQ ID NO:99)), SpyTag, a peptide which binds covalently to SpyCatcher protein (AHIVMVDAYKPTK (SEQ ID NO:100)), SnoopTag, a peptide which binds covalently to SnoopCatcher protein (KLGDIEFIKVNK (SEQ ID NO:101)), BCCP (Biotin Carboxyl Carrier Protein), a protein domain biotinylated by BirA enabling recognition by streptavidin, Glutathione-S-transferase-tag, a protein which binds to immobilized glutathione, Green fluorescent protein-tag, a protein which is spontaneously fluorescent and can be bound by nanobodies, HaloTag, a mutated bacterial haloalkane dehalogenase that covalently attaches to a reactive haloalkane substrate, this allows attachment to a wide variety of substrates., Maltose binding protein-tag, a protein which binds to amylose agarose, Nus-tag,

Thioredoxin-tag, Fc-tag, derived from immunoglobulin Fc domain, allow dimerization and solubilization. Can be used for purification on Protein-A Sepharose, Designed Intrinsically Disordered tags containing disorder promoting amino acids (P,E,S,T,A,Q,G,), and Ty-tag. In some embodiments, the polypeptide comprises a 6H tag of SEQ ID NO:87. II. Proteinaceous Compositions

The polypeptides or polynucleotides of the disclosure, such as the CBD, serum protein, therapeutic agent, masking agent, linker, or cytokine polypeptide, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-190.

The polypeptides of the disclosure, such as the CBD, serum protein, therapeutic agent, masking agent, linker, or cytokine polypeptide, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NOS:1-190.

In some embodiments, a polypeptide of the disclosure may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOS:1-190.

In some embodiments, a polypeptide of the disclosure, such as the CBD, serum protein, therapeutic agent, masking agent, linker, or cytokine polypeptide, may comprise at least, at most, about, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155,156, 157,158, 159, 160,161, 162,163, 164, 165,166, 167,168, 169,170, 171,172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOS:1-190.

In some embodiments, the polypeptide, such as the CBD, serum protein, therapeutic agent, masking agent, linker, or cytokine polypeptide, may comprise at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312,313, 314,315, 316, 317,318, 319,320, 321, 322,323, 324,325, 326,327, 328,329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOS:1-190 that are at least, at most, or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:1-190.

A polypeptide of the disclosure, such as the CBD, serum protein, therapeutic agent, masking agent, linker, or cytokine polypeptide may be at least, at most, or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) similar, identical, or homologous with one of SEQ ID NOS:1-190.

The polypeptides and nucleic acids of the disclosure may include at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498,499,500, 501,502, 503, 504,505, 506,507, 508, 509,510, 511,512, 513,514, 515,516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NOS:1-190. One or more of these substitutions may be specifically excluded from an embodiment.

Peptides, polypeptides, and proteins of the disclosure, such as the CBD, serum protein, therapeutic agent, masking agent, linker, or cytokine polypeptide, having at least, having at least, or having 70, 75, 80, 85, 90, 91, 92, 93, 94, 95, 96, 97, 98, 99, 100% identity to any one of SEQ ID NOS:1-190 includes a fragment of segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any range derivable therein) and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 (or any range derivable therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of:

alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. One or more of these substitutions may be specifically excluded from an embodiment.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Nucleic Acids

In certain embodiments, the current disclosure concerns recombinant polynucleotides encoding the proteins, polypeptides, and peptides of the invention, such as the

CBD, serum protein, therapeutic agent, masking agent, linker, or cytokine polypeptide, and/or other molecules. Therefore, certain embodiments relate to nucleotides encoding for a CBD, serum protein, therapeutic agent, masking agent, linker, or cytokine polypeptide, and fragments thereof.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences.

Polynucleotides may be single-stranded (coding or antisense) or double-stranded, and may be RNA, DNA (genomic, cDNA or synthetic), analogs thereof, or a combination thereof.

Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs (or any range derivable therein), including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other embodiments, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression.

Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991).

IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACKTM BACULOVIRUS EXPRESSION SYSTEM FROM CLONTECH®.

In addition to the disclosed expression systems of the invention, other examples of expression systems include STRATAGENE®' s COMPLETE CONTROL—Inducible Mammalian Expression System, which involves a synthetic ecdysone-inducible receptor, or its pET Expression System, an E. coli expression system. Another example of an inducible expression system is available from INVITROGEN®, which carries the T-REXTM (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. Additional Therapies

### A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

### 1. Checkpoint Inhibitors and Combination Treatment

Embodiments of the disclosure may include administration of immune checkpoint inhibitors (also referred to as checkpoint inhibitor therapy), which are further described below.

### a. PD-1, PD-L1, and PD-L2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-Ll activity.

Alternative names for “PD-1” include CD279 and SLEB2. Alternative names for “PD-Ll” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-Ll and/or PD-L2. In another embodiment, a PD-Ll inhibitor is a molecule that inhibits the binding of PD-Ll to its binding partners. In a specific aspect, PD-Ll binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and

US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-Ll inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab,

KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-Ll inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1

(CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-IBB (CD137; TNFRSF9), CD4OL (CD4OLG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFN(3), type II (IFNy) and type III (IFNX).

Interleukins have an array of immune system effects. IL-2 is an exemplary interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

In some embodiments, the additional therapy comprises neoantigen administration. Many tumors express mutations. These mutations potentially create new targetable antigens (neoantigens) for use in T cell immunotherapy. The presence of CD8+T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 doses (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or about 1, 2, 3, 4, 5, 6, 7, 8, 9, 10, 12, 14, 15, 20, 25, 30, 35, 40, 45, or 50 Gy (or any derivable range therein. In some embodiments, at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or about 1, 2, 3, 4, 5, 6, 7, 8, 9, 10, 11, or 12 (or any derivable range therein) fractionated doses are administered per day. In some embodiments, at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. Combination Therapy

The compositions and related methods of the present disclosure, particularly administration of the masked therapeutic agents of the disclosure may also be used in combination with the administration of additional therapies such as the additional therapeutics described herein or in combination with other traditional therapeutics known in the art for the treatment of cancer.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent or therapy described herein or known in the art is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some embodiments, more than one course of therapy may be employed. It is contemplated that multiple courses may be implemented.

### VI. Therapeutic Methods

The current methods and compositions relate to methods for treating cancer. In some embodiments, the cancer comprises a solid tumor. In some embodiments, the cancer is non-lymphatic. In some embodiments, the cancer is breast cancer or colon cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some embodiments, the administration is intratumoral or intralymphatic or peri-tumoral. In some embodiments, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer , leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous hi stiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VII. Pharmaceutical Compositions and Methods

In some embodiments, pharmaceutical compositions are administered to a subject. Different aspects involve administering an effective amount of a composition to a subject. In some embodiments, a composition comprising an inhibitor may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, transcatheter injection, intraarterial injection, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into cancer tissue, and injection into lymph nodes. In some embodiments, the administration is systemic.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### VIII. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLE 1

### Masked Cytokines Decrease Toxicity and Increase Tumor Specific Activation

Cytokine cancer immunotherapy using interleukin (IL)-12 has shown strong antitumor efficacy in both mouse and in human. However, due to its severe toxicity, some IL12 clinical trials have been terminated or unsuccessful. IL12 has not been approved to use in the clinic to date (1).

Immunotherapies serve to activate immune responses, and as such, side-effects typically result from drug action in healthy organs. One solution to overcome the problem of toxicity is to prevent cytokine action in healthy tissue. One solution involves transformation of a cytokine into a pro-drug that is inactive in healthy tissues and during systemic circulation but is activated locally at the site of disease. In an antibody format, this concept has been developed as a probody (2). IL12 fusion to an anti-IL12 antibody to cover the IL12 receptor binding site has been developed very recently, yet success with this approach involving antibody domains has not been shown in vivo (3). Described herein is a pro-cytokine (prodrug in cytokine format) comprising a masking agent and cytokine linked via a substrate peptide that is cleaved by an enzyme that is present in the tumor microenvironment (tumor-associated protease cleavage site), such as matrix metalloproteinases (MMPs). MMP is a protease family that is specifically activated within the tumor microenvironment to cleave its recognition site. The resulting pro-cytokine was shown to be locally activated in the tumor microenvironment, providing an opportunity to act as tumor-targeted immunotherapy.

IL12 is a heterodimeric glycosylated cytokine comprised of disulfide-linked p35 (˜35 kDa) and p40 (˜40 kDa) subunits. It is secreted as an early pro-inflammatory cytokine by activated antigen presenting cells (APCs) in response to infection. Upon binding to IL12 receptor on CD4+ T cells, IL12 promotes Thl polarization and results in IFNγ production (4). IL12 is considered to be an ideal antitumor therapeutic cytokine, as it activates both the innate and the adaptive arms of the immune system. Despite this, systemic administration of recombinant human IL12 (rh-IL12) showed unsatisfactory outcomes in clinical trials due to intolerable toxicity resulting in discontinuation of trials with systemic IL12 (5). A major barrier in recombinant IL12 therapy stems from the inability to reach sufficiently high local concentrations within the tumors, thus motivating for development of tumor-targeted recombinant IL12 therapy to unleash the full therapeutic potential of this cytokine (1). Here, the inventors sought to develop a binding site masking methodology to IL12, to improve its toxicity. The inevntors have used IL12 receptor beta 1 (IL12R(31) fibronectin I and II domains, which are the cytokine binding domain of this receptor protein, as a mask for IL12 binding site.

The inventors have shown that chemical conjugation of CBD protein to CPIs and recombinant fusion to IL-2 resulted in enhanced antitumor efficacy compared to their unmodified forms. The inventors have combined this CBD technology with the pro-cytokine format; this combination provides for the prolonged presence of the masked pro-cytokine in the enzymatic microenvironment to enhance enzymatic unmasking of the pro-cytokine. This approach is also applicable to probodies.

### A. Results

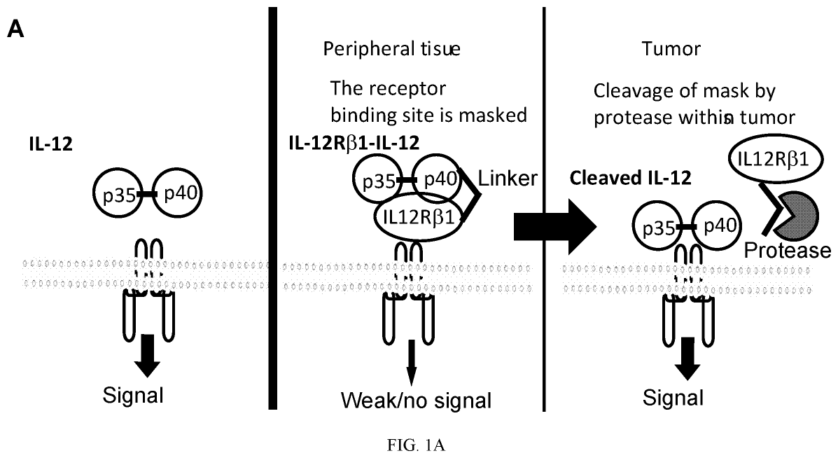

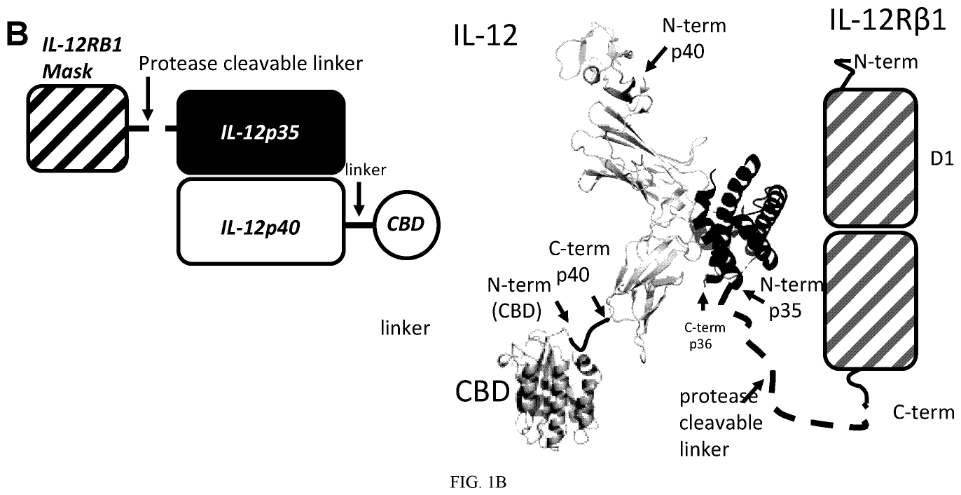

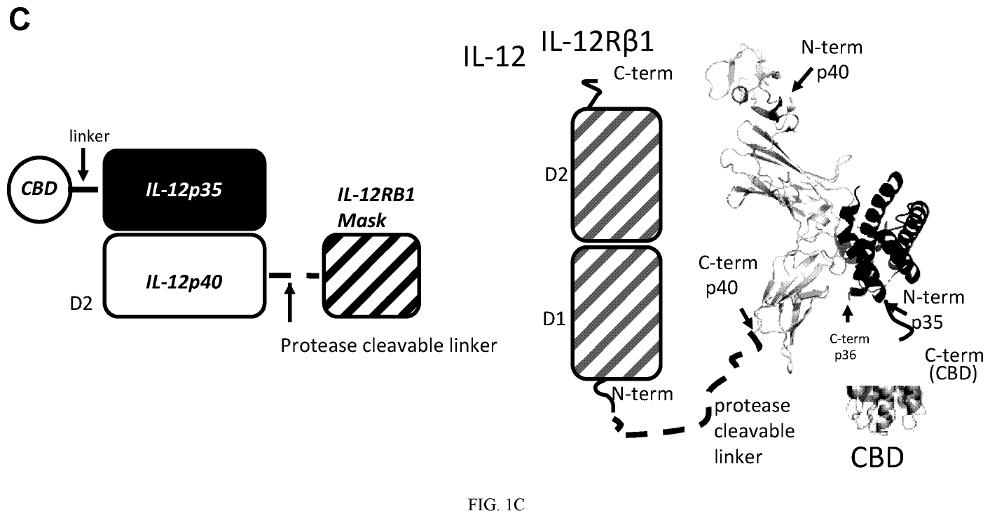

The inventors designed a IL12Rβ1 fusion to IL12 protein utilizing the first two fibronectin domains, D1 and D2, of the receptor (FIG. 1). Proteins were expressed by HEK293 cells and then purified by Histidine tag-affinity purification and size-exclusion column purification.

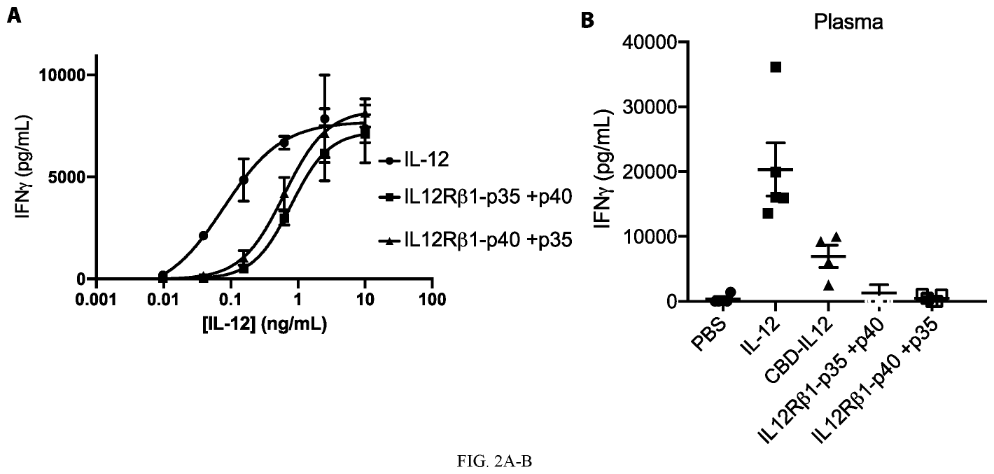

To test the activity of IL12Rβ1-IL12, splenocytes were cultured in the presence of IL12Rβ1-IL12 for 2 days in vitro. IL12Rβ1 fusion to IL12 (in both cases of IL12R(31 fused to p35 and p40) significantly decreased the concentration of IFNy, a main downstream molecule of IL12, in the culture media compared to IL12, suggesting that IL12Rβ1 fusion to IL12 decreases IL12 activity (FIG. 2A). The inventors measured blood plasma IFNγ concentration, because IFNγ is the main downstream molecule of IL12 immunological action and systemic IFNγ release causes systemic toxicity (FIG. 2B). IL12 and its variants were injected to B16F10 tumor bearing mice and blood plasma was collected 2 days after injection, when IFNγ level is the highest based on previous experiments. Without the protease cleavage sequence linking the IL12Rβ1 domain, IL12Rβ1-IL12 (in both cases of IL12R(31 fused to p35 and p40) showed an undetectable level of IFNγ in the plasma, whereas unmodified IL12 showed high IFNy concentration.

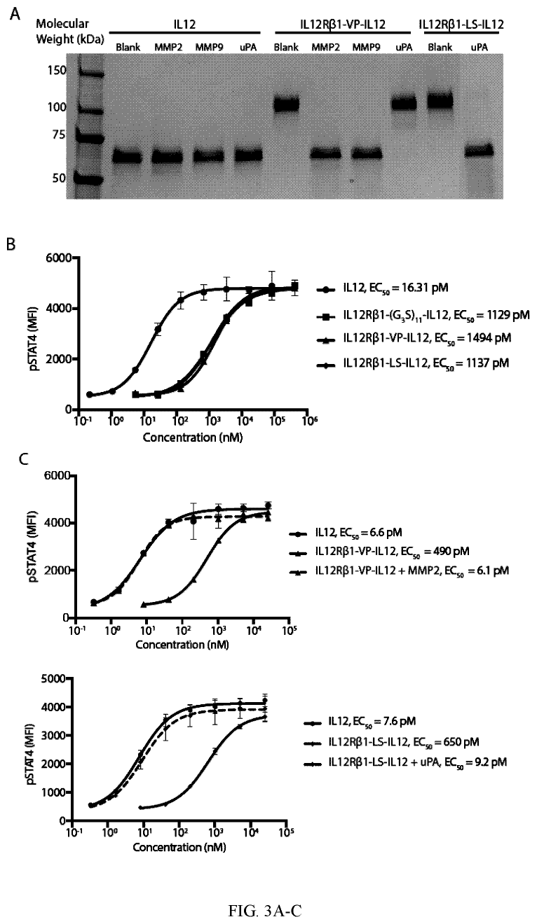

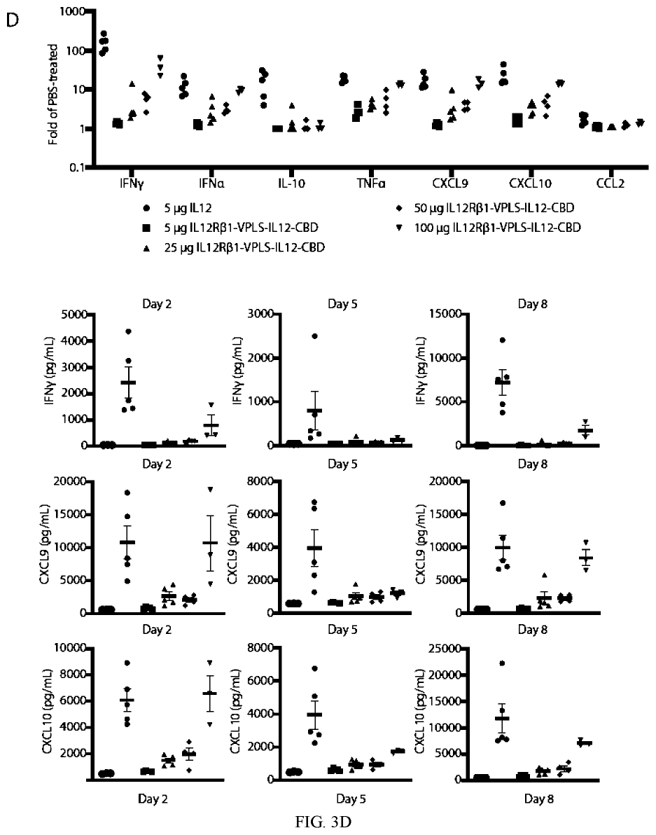

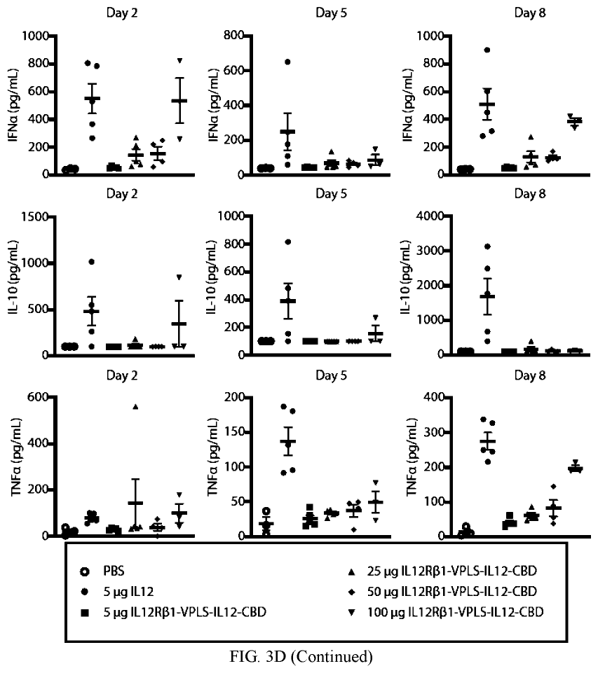

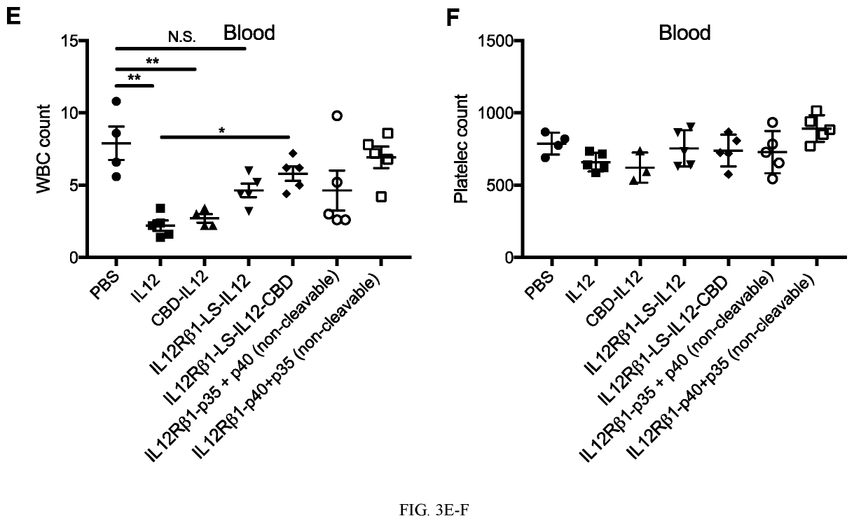

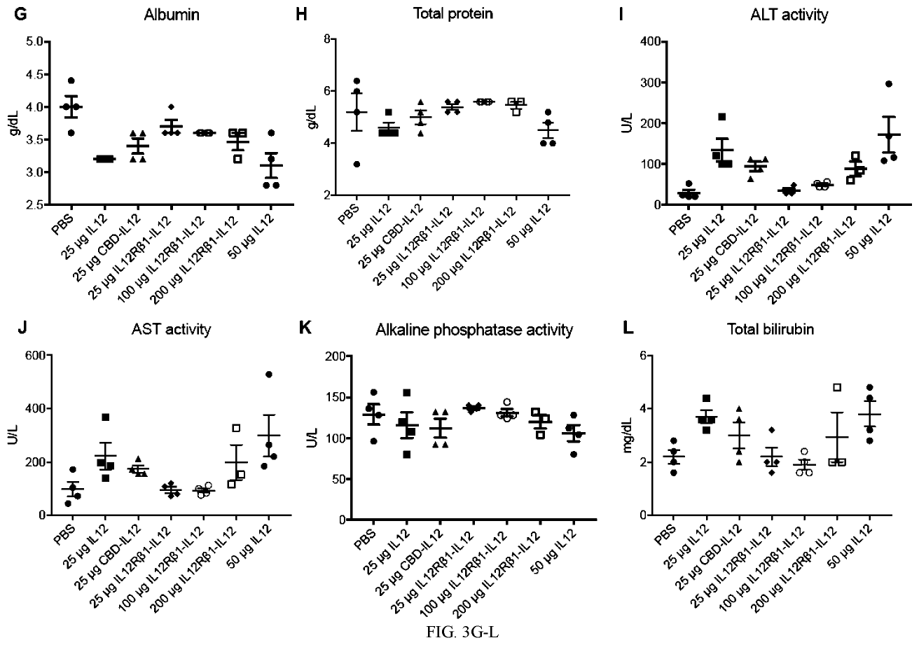

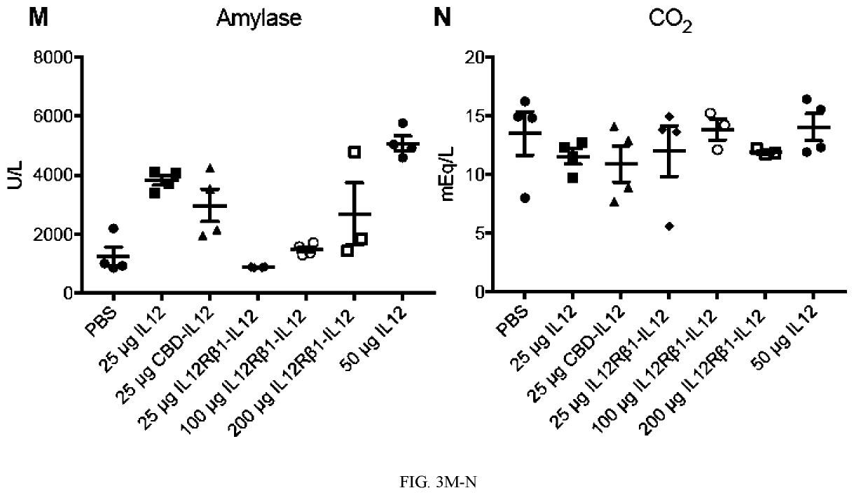

The inventors then expressed the IL12Rβ1-IL12 comprising the protease cleavage site inserted between IL12Rβ1 and IL12 to achieve tumor specific IL12 activation. The inventors tested whether VPLSLYS (SEQ ID NO:50)-containing IL12Rβ1-VP-IL12 can be proteolytically cleaved by VPLSLYS (SEQ ID NO:50)-recognizing enzymes MMP2 and MMP9 (FIG. 3A). SDS-PAGE analysis revealed that MMP2 and MMP9 fully cleaved the IL12Rβ1 portion, yielding IL12 heterodimer (-60 kDa). Similarly, IL12Rβ1-LS-IL12 containing LSGRSDNH (SEQ ID NO:49) substrate was cleaved by uPA. These enzymes had no effect on IL12 itself as shown in FIG. 3A.

Next, inventors tested whether fusion of IL12Rβ1 with the p35 chain was able to reduce the bioactivity of IL12 (FIG. 3B). As a proxy of IL12 bioactivity, the inventors measured STAT4 phosphorylation in response to varying amounts of the cytokine. The ECso value of the unmodified IL12 was 16.3 pM, whereas that of proIL12 constructs was —80-fold less. To determine whether treatment of IL12Rβ1-VP-IL12 with MMP2 and IL12Rβ1-LS-IL12 with uPA can yield a functional response on T cells, the inventors compared the bioactivity of cleaved and noncleaved constructs to that of IL12 (FIG. 3C). In accordance with previous results, noncleaved constructs exhibited —80-fold decrease in STAT4 phosphorylation. Yet, treatment of the proIL12 constructs with respective proteases fully recovered IL12 bioactivity, displaying a similar dose-response relationship as unmodified IL12.

To determine whether IL12Rβ1-VPLS-IL12-CBD also reduces the toxicity in vivo, the inventors dosed healthy mice with either unmodified IL12 or escalating doses of IL12Rβ1-VPLS-IL12-CBD 3 times, every 3 days (FIG. 3D). 2 days after each injection, mice were bled, and sera were analyzed for the presence of proinflammatory biomarkers using LEGENDplex technology. The data indicate that IL12Rβ1-VPLS-IL12-CBD exhibited an enhanced safety profile when compared to IL12. Importantly, 5μg of IL12Rβ1-VPLS-IL12-CBD did not appreciably upregulate any of the toxic markers when compared to PB S-treated mice and 100 i_tg of IL12Rβ1-VPLS-IL12-CBD was generally tolerated to a greater extent when compared to IL12-treated mice. Next, the blood cell count 2 days after IL12 injection in B16F10 tumor bearing mice was tested. IL12 and CBD-IL12 induced the decrease of white blood cell count in the blood, whereas all variants of IL12Rβ1-IL12 did not (FIG. 3E). The platelet count was maintained in all cases. These data suggest that IL12Rβ1 fusion to IL12 decreases systemic toxicity (FIG. 3F). Blood toxicity markers were analyzed by a biochemistry analyzer 3 days after IL12Rβ1-IL12 injection to non-tumor bearing mice. When 25 μg or 100 μg of IL12Rβ1-IL12 was injected, liver damage markers (blood albumin concentration, total protein, alanine aminotransferase (ALT) activity, aspartate aminotransferase (AST) activity, and alkaline phosphate activity), kidney damage marker (total bilirubin), pancreas damage marker (amylase), lung damage marker (CO₂ concentration) were comparable to PBS injected group (FIG. 3G-N). 25 μg or 50 μg of IL12 injected mice have shown marked decrease in blood albumin concentration and increased ALT activity, AST activity, total bilirubin, and amylase, suggesting toxicity to several organs. When 200 μg of IL12Rβ1-IL12 was injected, one mouse out of three has responded to elevate AST activity, total bilirubin, and amylase. These data suggest that maximum tolerated dose of IL12Rβ1-IL12 is at least more than 100 μg. When inventors combined IL12Rβ1-IL12 and anti-PD-1 blocking antibody, ALT activity and amylase levels are not dramatically increased compared to wild-type IL12 therapy (FIG. 30P).

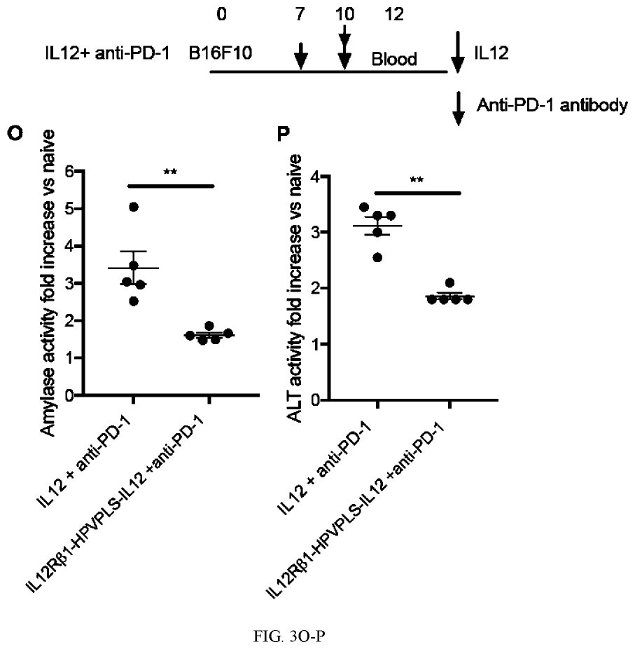

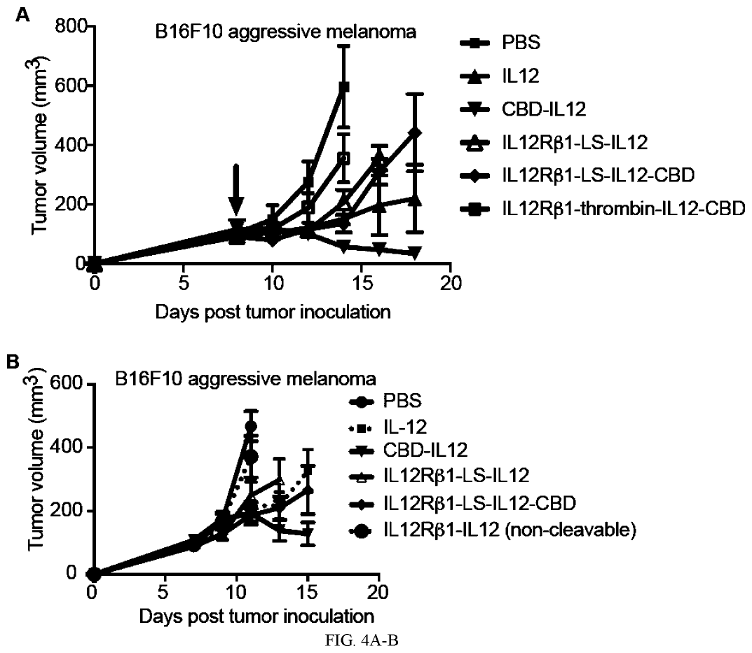

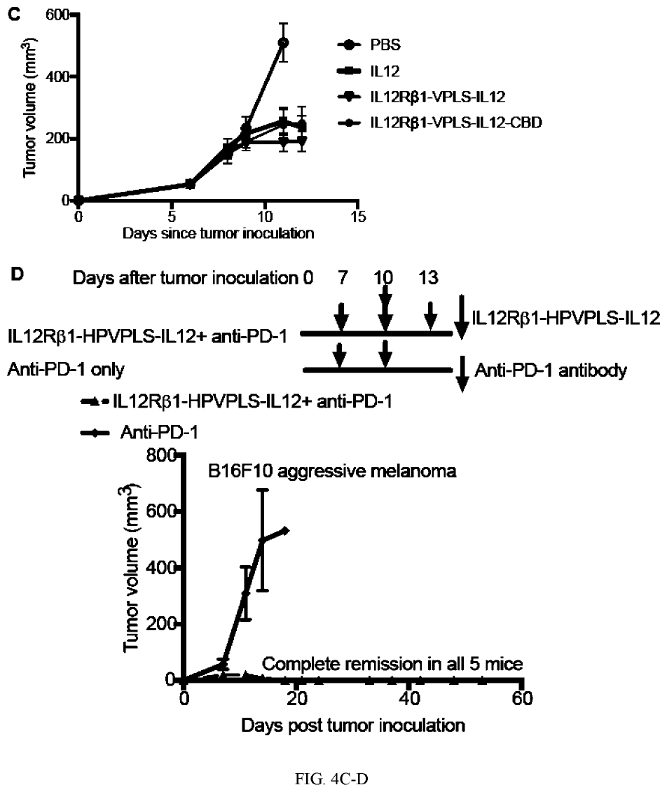

The inventors then tested antitumor efficacy of IL12Rβ1 fused IL12 using Bl6F10 model (FIG. 4). IL12Rβ1-IL12 with uPA and thrombin cleavage site showed antitumor effects. In contrast, IL12Rβ1-IL12 without cleavable sequence did not show anti-tumor efficacy (FIG. 4B). These data demonstrate that IL12Rβ1-IL12 is activated within the tumor by the protease and show antitumor efficacy. Moreover, the anti-tumor efficacy of IL12Rβ1-IL12 comprising the CBD demonstrated higher anti-tumor efficacy than the variant without the CBD, demonstrating that the effect of the CBD in prolonging the presence of the pro-cytokine in the enzymatic environment of the tumor enhances its activation. The inventors also tested lower dose of IL12Rβ1-VPLS-IL12 and found that both IL12Rβ1-VPLS-IL12 and IL12Rβ1-VPLS-IL12-CBD are as efficacious as IL12 (FIG. 4C). The inventors next tested if IL12Rβ1-IL12 with uPA and MMP cleavage site synergizes with anti-PD-1 blocking therapy (FIG. 4D). Anti-PD-1 antibody therapy did not cure any Bl6F10 mice, but the combination therapy of IL12Rβ1-LSHPVP-IL12 and anti-PD-1 antibody cured all five B16F10 tumors. Consequently, we found that IL12R131-IL12 increases the therapeutic effect of anti-PD-1 therapy.

### B. Conclusion

Cytokines are key factors for antitumor activities, but not many of them have been translated to the clinic to date. IL12 is one of the strongest antitumor cytokines, but due to its high toxicity, the clinical trial has been terminated or unsuccessful. Thus, decreasing its toxicity is an important strategy to translate it to the clinic. To improve CBD-IL12 therapy, the inventors fused a domain of the IL12 receptor IL12Rβ1 to the IL12, to form IL12Rβ1-IL12. This fusion is inactive, but the inclusion of an MMP or thrombin cleavage site between the receptor mask and the cytokine yields a pro-cytokine that can be activated in the tumor microenvironment. The inventors have demonstrated that the immunotoxicity of the IL12 is thus reduced, and that the IL12Rβ1-IL12 fusion with the protease-sensitive linker retains therapeutic utility.

Because it was observed that single repeat of MMP or thrombin cleavage site between the receptor mask and the cytokine gave an antitumor efficacy, it was hypothesized that introducing multi-cleavage sites in the linker (e.g. tandem MMP, tandem thrombin, and MMP-thrombin dyads and repeats) would increase the protease sensitivity and might increase the antitumor efficacy of IL12Rβ1-IL12 therapy.

In conclusion, described herein is the development of a technology to reduce cytokine toxicity by fusing the cytokine receptor to the cytokine. Tumor specific proteases cleave the linker to activate the cytokine within the tumor.

### C. Materials and Methods

### 1. Production and Purification of the Recombinant Fusion Protein of VWF A3 domain, IL12Rβ1 and IL12

Protein production and purification were performed as described previously (7). The sequence encoding for the human VWF A3 domain residues Cys1670-Glyl874 (907-1111 of mature VWF), mouse IL12, IL12Rβ1, the fusion proteins were synthesized and subcloned into the mammalian expression vector pcDNA3.1(+) by Genscript. A sequence encoding for 6 His was added at the N-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/ml. 2 μg/m1 plasmid DNA, 2 μg/m1 linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO2. 7 days after transfection, the cell culture medium was collected by centrifugation and filtered through a 0.22 p.m filter. Culture media was loaded onto a HisTrap HP 5 ml column (GE Healthcare), using an AKTA pure 25 (GE Healthcare). After washing of the column with wash buffer (20 mM imidazole, 20 mM NaH₂PO₄, 0.5 M NaCl, pH 7.4), the protein was eluted with a gradient of 500 mM imidazole (in 20 mM NaH₂PO₄, 0.5 M NaCl, pH 7.4). The elusion solution was further purified with size exclusion chromatography using a HiLoad Superdex 200PG column (GE healthcare). All purification steps were carried out at 4° C. The expression of laminin LG domain was determined by western blotting using anti-His tag antibody (BioLegend) and the proteins were verified as >90% pure by SD S-PAGE.

| | |
|---|---|
| VWF A3 | SEQ ID NO: 1 |
| domain | |
| protein | |
| mIL12Rβ1 | SEQ ID NO: 2 |
| Human IL12 | p35 Subunit: SEQ ID NO: 3 |
| | p40 Subunit: SEQ ID NO: 4 |
| Mouse IL12 | p35 Subunit: SEQ ID NO: 5 |
| | p40 Subunit: SEQ ID NO: 6 |
| mIL12Rβ1- | QLGASGPGDGCCVEKTSFPEGASGSPLGPRNLSCYRV |
| Thrombin- | SKTDYECSWQYDGPEDNVSHVLWCCFVPPNHTHTGQE |
| IL12p35-6H | RCRYFSSGPDRTVQFWEQDGIPVLSKVNFWVESRLGN |
| | RTMKSQKISQYLYNWTKTTPPLGHIKVSQSHRQLRMD |
| | WNVSEEAGAEVQFRRRMPTTNWTLGDCGPQVNSGSGV |
| | LGDIRGSMSESCLCPSENMAQEIQIRRRRRLSSGAPG |
| | GPWSDWSNIPVCVPPEVLPQALVPRGSGGGSGGGSRV |
| | IPVSGPARCLSQSRNLLKTTDDMVKTAREKLKHYSCT |
| | AEDIDHEDITRDQTSTLKTCLPLELHKNESCLATRET |
| | SSTTRGSCLPPQKTSLMMTLCLGSIYEDLKMYQTEFQ |
| | AINAALQNHNHQQIILDKGMLVAIDELMQSLNHNGET |
| | LRQKPPVGEADPYRVKMKLCILLHAFSTRVVTINRVM |
| | GYLSSAHHHHHH (SEQ ID NO: 103) |
| CBD- | SEQ ID NO: 8 |
| mIL12Rβ1- | |
| Thrombin- | |
| IL12p40 | |
| mIL12Rβ1- | QLGASGPGDGCCVEKTSFPEGASGSPLGPRNLSCYRV |
| LS- | SKTDYECSWQYDGPEDNVSHVLWCCFVPPNHTHTGQE |
| IL12p35- | RCRYFSSGPDRTVQFWEQDGIPVLSKVNFWVESRLGN |
| 6H | RTMKSQKISQYLYNWTKTTPPLGHIKVSQSHRQLRMD |
| | WNVSEEAGAEVQFRRRMPTTNWTLGDCGPQVNSGSGV |
| | LGDIRGSMSESCLCPSENMAQEIQIRRRRRLSSGAPG |
| | GPWSDWSMPVCVPPEVLPQAGLLSGRSDNHGGGSGGG |
| | SRVIPVSGPARCLSQSRNLLKTTDDMVKTAREKLKHY |
| | SCTAEDIDHEDITRDQTSTLKTCLPLELHKNESCLAT |
| | RETSSTTRGSCLPPQKTSLMMTLCLGSIYEDLKMYQT |
| | EFQAINAALQNHNHQQIILDKGMLVAIDELMQSLNHN |
| | GETLRQKPPVGEADPYRVKMKLCILLHAFSTRVVTIN |
| | RVMGYLSSAHHHHHH (SEQ ID NO: 104) |
| mIL12Rβ1- | SEQ ID NO: 10 |
| LS-linker- | |
| IL12p40 | |
| CBD- | SEQ ID NO: 11 |
| mIL12Rβ1- | |
| Thrombin- | |
| IL12p40 | |
| CBD- | SEQ ID NO: 12 |
| mIL12Rβ1- | |
| LS- | |
| IL12p40 | |
| MMP | SEQ ID NO: 13 |
| responsive | |
| sequence | |
| Thrombin | SEQ ID NO: 14 |
| responsive | |
| sequence | |
| mIL12Rβ1- | QLGASGPGDGCCVEKTSFPEGASGSPLGPRNLSCYRV |
| Thrombin- | SKTDYECSWQYDGPEDNVSHVLWCCFVPPNHTHTGQE |
| LS-LS- | RCRYFSSGPDRTVQFWEQDGIPVLSKVNFWVESRLGN |
| inker- | RTMKSQKISQYLYNWTKTTPPLGHIKVSQSHRQLRMD |
| IL12p35- | WNVSEEAGAEVQFRRRMPTTNWTLGDCGPQVNSGSGV |
| 6H | LGDIRGSMSESCLCPSENMAQEIQIRRRRRLSSGAPG |
| | GPWSDWSMPVCVPPEVLPQALVPRGSGLLSGRSDNHG |
| | LLSGRSDNHGGGSGGGSRVIPVSGPARCLSQSRNLLK |
| | TTDDMVKTAREKLKHYSCTAEDIDHEDITRDQTSTLK |
| | TCLPLELHKNESCLATRETSSTTRGSCLPPQKTSLMM |
| | TLCLGSIYEDLKMYQTEFQAINAALQNHNHQQIILDK |
| | GMLVAIDELMQSLNHNGETLRQKPPVGEADPYRVKMK |
| | LCILLHAFSTRVVTINRVMGYLSSAHHHHHH |
| | (SEQ ID NO: 105) |
| mIL12Rβ1- | CCVEKTSFPEGASGSPLGPRNLSCYRVSKTDYECSWQ |
| LSLSLS- | YDGPEDNVSHVLWCCFVPPNHTHTGQERCRYFSSGPD |
| IL12p35- | RTVQFWEQDGIPVLSKVNFWVESRLGNRTMKSQKISQ |
| 6H | YLYNWTKTTPPLGHIKVSQSHRQLRMDWNVSEEAGAE |
| | VQFRRRMPTTNWTLGDCGPQVNSGSGVLGDIRGSMSE |
| | SCLCPSENMAQEIQIRRRRRLSSGAPGGPWSDWSMPV |
| | CVPPEVLPQAGLLSGRSDNHGLLSGRSDNHGLLSGRS |
| | DNHGGGSGGGSRVIPVSGPARCLSQSRNLLKTTDDMV |
| | KTAREKLKHYSCTAEDIDHEDITRDQTSTLKTCLPLE |
| | LHKNESCLATRETSSTTRGSCLPPQKTSLMMTLCLGS |
| | IYEDLKMYQTEFQAINAALQNHNHQQIILDKGMLVAI |
| | DELMQSLNHNGETLRQKPPVGEADPYRVKMKLCILLH |
| | AFSTRVVTINRVMGYLSSAHHHHHH (SEQ ID |
| | NO: 106) |
| mIL12Rβ1- | CCVEKTSFPEGASGSPLGPRNLSCYRVSKTDYECSWQ |
| Thrombin- | YDGPEDNVSHVLWCCFVPPNHTHTGQERCRYFSSGPD |
| Thrombin- | RTVQFWEQDGIPVLSKVNFWVESRLGNRTMKSQKISQ |
| Thrombin- | YLYNWTKTTPPLGHIKVSQSHRQLRMDWNVSEEAGAE |
| IL12p35- | VQFRRRMPTTNWTLGDCGPQVNSGSGVLGDIRGSMSE |
| 6H | SCLCPSENMAQEIQIRRRRRLSSGAPGGPWSDWSMPV |
| | CVPPEVLPQALVPRGSLVPRGSLVPRGSGGGSGGGSR |
| | VIPVSGPARCLSQSRNLLKTTDDMVKTAREKLKHYSC |
| | TAEDIDHEDITRDQTSTLKTCLPLELHKNESCLATRE |
| | TSSTTRGSCLPPQKTSLMMTLCLGSIYEDLKMYQTEF |
| | QAINAALQNHNHQQIILDKGMLVAIDELMQSLNHNGE |
| | TLRQKPPVGEADPYRVKMKLCILLHAFSTRVVTINRV |
| | MGYLSSAHHHHHH (SEQ ID NO: 107) |
| mIL12Rβ1- | CCVEKTSFPEGASGSPLGPRNLSCYRVSKTDYECSWQ |
| Throm3- | YDGPEDNVSHVLWCCFVPPNHTHTGQERCRYFSSGPD |
| MMP3linker- | RTVQFWEQDGIPVLSKVNFWVESRLGNRTMKSQKISQ |
| IL12p35- | YLYNWTKTTPPLGHIKVSQSHRQLRMDWNVSEEAGAE |
| 6H | VQFRRRMPTTNWTLGDCGPQVNSGSGVLGDIRGSMSE |
| | SCLCPSENMAQEIQIRRRRRLSSGAPGGPWSDWSMPV |
| | CVPPEVLPQALVPRGSLVPRGSLVPRGSGLLSGRSDN |
| | HGLLSGRSDNHGLLSGRSDNHGGGSGGGSRVIPVSGP |
| | ARCLSQSRNLLKTTDDMVKTAREKLKHYSCTAEDIDH |
| | EDITRDQTSTLKTCLPLELHKNESCLATRETSSTTRG |
| | SCLPPQKTSLMMTLCLGSIYEDLKMYQTEFQAINAAL |
| | QNHNHQQIILDKGMLVAIDELMQSLNHNGETLRQKPP |
| | VGEADPYRVKMKLCILLHAFSTRVVTINRVMGYLSSA |
| | HHHHHH (SEQ ID NO: 108) |
| Human | SEQ ID NO: 19 |
| IL12Rβ1 | |
| Mouse | SEQ ID NO: 20 |
| IL12Rβ1 | |
| Human | SEQ ID NO: 21 |
| IL12Rβ2 | |
| Mouse | SEQ ID NO: 22 |
| IL12Rβ2 | |

### 2. Pro-IL12 Cleavage Assays

Recombinant mouse matrix metalloproteinase-2 (MMP2), MMP9 and recombinant human urokinase plasminogen activator (uPA) were purchased from R&D. Since MMP2 and MMP9 were supplied in their zymogen form, MMPs were first activated using 1 mM p-aminophenylmercuric acetate (APMA, Sigma) for 2 hr at 37° C. Following activation, MMPs and cytokines were diluted in an assay buffer containing 150 mM NaCl, 50 mM Tris, 10 mM CaCl₂, 0.05% Brij-35 at pH=7.5. Final concentrations of MMP2, MMP9 and cytokine were 2 μg/mL, 5 μg/mL and 50 μg/mL, respectively. Cleavage conducted for 30 min at 37° C. Samples were then analyzed via gel electrophoresis. Cleavage using uPA was conducted according to manufacturer's protocol. Concentration of uPA was 10 μg/mL

### 3. In vitro Stimulation Assay

Mouse CD8⁺ T cells were purified from spleens of C57BL/6 mice using EasySep mouse CD8⁺ T cell isolation kit (Stem Cell). Purified CD8⁺ T cells (10⁶ cells/mL) were activated in six-well plates precoated with 2 μg,/mL a-CD3 (clone 17A2, Bioxcell) and supplemented with soluble 5 μg/mL a-CD28 (clone 37.51, BioLegend) and 30 ng/mL mouse

IL-2 (Peprotech) for 3 days. Culture medium was IMDM (Gibco) containing 10% heat-inactivated FBS, 1% Penicillin/Streptomycin and 50μM 2-mercaptoethanol (Sigma Aldrich). After 3 days of culture, activated CD8⁺ T cells were rested for 6 hrs in fresh culture medium and were transferred into 96-well plates (50,000 cells/well). Indicated amounts of IL-12 or proIL12 variants were applied to CD8⁺ T cells for 20 min at 37° C. to induce STAT4 phosphorylation. Cells were fixed immediately using BD Phosflow Lyse/Fix buffer for 10 min at 37° C. and then permeabilized with BD Phosflow Perm Buffer III for 30 min on ice. Cells were stained with Alexa Fluor (AF) 647-conjugated antibody against pSTAT4 (clone 38, BD) recognizing phosphorylation of Tyr693. Staining was performed for 1 hr at room temperature (RT) in the dark. Cells were acquired on BD Fortessa X-20 and data were analyzed using FlowJo (Treestar). Mean Fluorescence Intensity (MFI) of pSTAT4⁺ population was plotted against cytokine concentration. Dose-response curve was fitted using Prism (v8, GraphPad).

### 4. Mice and Cell Lines

The mice and cell lines were prepared as described previously (8). C57BL/6 age 8 to 12 weeks, were obtained from the Charles River laboratories. Experiments were performed with approval from the Institutional Animal Care and Use Committee of the University of Chicago. B16F10 cells were obtained from the American Type Culture Collection and cultured according to the instructions. All cell lines were checked for mycoplasma contamination by a pathogen test IMPACT I (IDEXX BioResearch).

### 5. Plasma Cytokine Concentration Analysis

The measurement is performed as described previously (8). 5×10⁵ B16F10 melanoma cells were injected intradermally on left side of the back of each 9 week old C57BL/6 mouse. After 7 or 8 days, mice received 25 μg of IL12 and equimolar of IL12 variants. 100 μg of anti-PD-1 antibody (clone:29F.1A12, BioXCell) was injected i.p. 2 days after IL12 injection, blood samples were collected in heparinized tubes containing EDTA, followed centrifugation. Cytokine concentration in plasma was measured by Ready-SET-Go!

ELISA kits (eBioscience) or LEGENDplex kit (BioLegend) according to the manufacture's protocol. BD Fortessa X-20 flow cytometry system and FlowJo was used to analyze LEGENDplex results.

### 6. Toxicity Marker Analysis in Serum

9 week old C57BL/6 mouse were used. Mice received IL12 and IL12 variants i.v.

3 days after IL12 injection, blood samples were collected in tubes, followed by clotting and centrifugation. Serum damage markers were measured by Vet Axcel (Alfa Wassermann) according to the manufacture's protocol.

### 7. Anti-tumor efficacy of IL12Rβ1-IL12 on B16F10 Tumor

The measurement is performed as described previously (8). A total of 5×10⁵ B16F10 cells re-suspended in 50 μL of PBS were inoculated intradermally on the left side of the back of each C57BL/6 mouse. After 7, 8, and/or 10 days, mice were injected with IL12 (25 μg), IL12R01-IL12 (equimolar, or 100 μg IL12 based), or CBD-IL12 (equimolar) i.v. 100 μg of anti-PD-1 antibody (clone:29F.1A12, BioXCell) was injected i.p. on days 7, 10 and/or13. Tumors were measured with a digital caliper starting 8 days after tumor inoculation, and volumes were calculated as ellipsoids, where V=4/3×3.14×depth/2×width/2×height/2. Mice were sacrificed at the point when either tumor volume had reached over 500 mm³.

### EXAMPLE 2

### Cytokine Embodiments

The technique of covering the receptor binding site by fusing a cytokine receptor domain can apply to other antitumor cytokines. This example teaches the receptor fusion to IL-2 and IFNγ to make other pro-cytokines, which are pro-IL-2 and pro-IFNγ. In all versions, MMP and/or thrombin responsive cleavage site are inserted between the receptor and cytokine. Exemplary embodiments of cytokines and masking agents are provided below:

| Cytokine | Masking Agent |
|---|---|
| Human IL-2 | human IL-2Ralpha |
| Human IL-2 | human IL-2Rbeta |
| Human IL-2 | human IL-2Rgamma |
| Mouse IL-2 | human IL-2Ralpha |
| Mouse IL-2 | human IL-2Rbeta |
| Mouse IL-2 | human IL-2Rgamma |
| Human IFNγ | human IFNγR1 |
| Human IFNγ | human IFNγR2 |
| Mouse IFNγ | mouse IFNγR1 |
| Mouse IFNγ | mouse IFNγR2 |

### EXAMPLE 3

### Addition of Serum Proteins for Prolonged Circulation

Pro-cytokine can be improved by CBD-fusion to yield prolonged residence in tumors and/or albumin fusion to yield prolonged circulation. Cytokines generally have a very short half-life in the blood (9). Because pro-cytokine technology is relying on the protease within the body (i.e. tumor), it is important to increase the retention time of injected pro-cytokine within tumor. The inventors employ two approaches to improve the CBD-cytokine platform. The first step is to fuse collagen binding domain to the pro-cytokines. As described in Example 1, CBD can target and retain the fused protein within the tumor due to the nature of the tumor vasculature. Thus, the activity of CBD-pro-cytokine is more specific within the tumor, resulting in enhanced efficacy and safety. This is a form of a dual tumor targeting system.

Another step is to extend the pro-cytokine blood half-life. Because extended blood half-life of injected cytokines will allow more chance to contact tumor tissues, it is hypothesized that the efficacy of CBD-pro-cytokines would be further enhanced. This can be achieved by fusing albumin to CBD-pro-cytokine or pro-cytokine. Thus, these additional embodiments of a tumor targeted cytokine with extended blood half-life, which is active only within the tumor microenvironment, are further contemplated.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references and the publications referred to throughout the specification, to the extent that they provide exemplary procedural or other details supplementary to those set forth herein, are specifically incorporated herein by reference.
1. Kerkar, S. P., et al. IL12 triggers a programmatic change in dysfunctional myeloid-derived cells within mouse tumors. The Journal of clinical investigation 121, 4746-4757 (2011).
2. Desnoyers, L. R., et al. Tumor-specific activation of an EGFR-targeting probody enhances therapeutic index. Sci Transl Med 5, 207ra144 (2013).
3. Skrombolas, D., Sullivan, M. & Frelinger, J.G. Development of an Interleukin-12 Fusion Protein That Is Activated by Cleavage with Matrix Metalloproteinase 9. Journal of interferon & cytokine research: the official journal of the International Society for Interferon and Cytokine Research 39, 233-245 (2019).
4. Rogge, L., et al. Selective expression of an interleukin-12 receptor component by human T helper 1 cells. The Journal of experimental medicine 185, 825-831 (1997).
5. Lasek, W., Zagozdzon, R. & Jakobisiak, M. Interleukin 12: still a promising candidate for tumor immunotherapy? Cancer immunology, immunotherapy: CII 63, 419-435 (2014).
6. Ishihara, J., et al. Targeted antibody and cytokine cancer immunotherapies through collagen affinity. Sci Transl Med 11(2019).
7. Martino, M. M., et al. Growth factors engineered for super-affinity to the extracellular matrix enhance tissue healing. Science 343, 885-888 (2014).
8. Ishihara, J., et al. Matrix-binding checkpoint immunotherapies enhance antitumor efficacy and reduce adverse events. Sci Transl Med 9(2017).
9. Zhu, E. F., et al. Synergistic innate and adaptive immune response to combination immunotherapy with anti-tumor antigen antibodies and extended serum half-life IL-2. Cancer Cell 27, 489-501 (2015)

1. A polypeptide comprising a cytokine linked to a masking agent through a linker, wherein the linker comprises one or more tumor-associated protease cleavage sites, and wherein the masking agent comprises a cytokine receptor polypeptide or fragment thereof that specifically binds to the cytokine.

2. The polypeptide of claim 1, wherein the cytokine comprises IL12 and wherein the masking agent comprises IL12R polypeptide or an IL12-binding fragment thereof.

3. The polypeptide of claim 2, wherein the IL12 comprises one or both of the p35 and p40 subunits.

4. The polypeptide of claim 3, wherein the IL12 comprises the p35 and p40 subunits linked through a disulfide bond.

5. The polypeptide of claim 3, wherein the IL12 comprises the p35 and p40 subunits linked through a peptide linker.

6. The polypeptide of any one of claims 2-5, wherein the IL12R polypeptide or fragment comprises IL12β1, or a fragment thereof.

7. The polypeptide of claim 6, wherein the IL12β1 polypeptide comprises one or both of fibronectin domains D1 and D2.

8. The polypeptide of any one of claims 2-7, wherein the masking agent is fused to the N-terminus of the p35 subunit of IL12, and wherein the linker is between the masking agent and the p35 subunit of IL12.

9. The polypeptide of any one of claims 2-7, wherein the masking agent is fused to the C-terminus of the p40 subunit of IL12, and wherein the linker is between the masking agent and the p40 subunit of IL12.

10. The polypeptide of claim 1, wherein the cytokine comprises IL-2 and the masking agent comprises IL-2Ra, IL-2R13, fragments, or combinations of fragments thereof.

11. The polypeptide of claim 1, wherein the cytokine comprises IFNγ and the masking agent comprises IFNγR1, IFNγR2, fragments, or combinations of fragments thereof.

12. The polypeptide of any one of claims 1-11, wherein the polypeptide comprises at least two tumor-associated protease cleavage sites.

13. The polypeptide of any one of claims 1-12, wherein the tumor-associated protease cleavage site comprises a uPA, matrix metalloproteinase, or thrombin cleavage site.

14. The polypeptide of any one of claims 1-13, wherein the cytokine comprises a pro-inflammatory cytokine.

15. The polypeptide of any one of claims 1-14, wherein the polypeptide is conjugated to a tumor targeting agent.

16. The polypeptide of claim 16, wherein the tumor targeting agent comprises an antibody or an antigen-binding fragment thereof.

17. The polypeptide of claim 16, wherein the antibody or antigen-binding fragment comprises a stroma targeting antibody or stroma-binding fragment thereof.

18. The polypeptide of claim 17, wherein the antibody or binding fragment specifically binds to fibronectin, alternatively spliced domains of fibronectin, collagens, tenascins, periostins, syndecans, proteoglycans, or a tumor stroma cell-specific antigen.

19. The polypeptide of claim 18, wherein the antibody or binding fragment specifically binds toe extra domain A (EDA) or extra domain B (EDB) of fibronectin.

20. The polypeptide of claim 18, wherein the tumor targeting agent comprises a Fab that specifically binds to an alternatively spliced domain of fibronectin comprising extra domain A (EDA).

21. The polypeptide of claim 16, wherein the tumor targeting agent comprises an antibody or antigen binding fragment thereof that specifically binds to a tumor-associated antigen.

22. The polypeptide of claim 15, wherein the tumor targeting agent comprises a collagen binding domain.

23. The polypeptide of claim 22, wherein the polypeptide comprises at least two collagen binding domains.

24. The polypeptide of claim 22 or 23, wherein the polypeptide comprises a collagen binding domain from decorin or von Willebrand factor (VWF).

25. The polypeptide of any one of claims 1-24, wherein the polypeptide further comprises a serum protein conjugated to the polypeptide.

26. The polypeptide of claim 25, wherein the serum protein is conjugated to the polypeptide through a peptide bond.

27. The polypeptide of claim 25 or 26, wherein the serum protein comprises albumin.

28. The polypeptide of any one of claims 1-27, wherein the polypeptide comprises a second linker.

29. The polypeptide of claim 28, wherein the second linker comprises glycine and serine amino acid residues.

30. The polypeptide of claim 29, wherein the linker comprises SEQ ID NO:47 or SEQ ID NO:48.

31. The polypeptide of any one of claims 1-30, wherein the polypeptide comprises a protein tag.

32. The polypeptide of any one of claims 1-31, wherein the polypeptide is not operatively linked to a particle, nanovesicle, or liposome.

33. A composition comprising the polypeptide of any one of claims 1-32.

34. The composition of claim 33, wherein the composition does not comprise a liposome, particle, or nanovesicle.

35. A nucleic acid encoding for the polypeptide of any one of claims 1-32.

36. A host cell comprising the nucleic acid of claim 35.

37. A method for making a polypeptide comprising expressing the nucleic acid of claim 35 in a cell and isolate the expressed polypeptide.

38. A method for treating cancer comprising administering the polypeptide of any one of claims 1-32 or the composition of claim 33 or 34.

39. The method of claim 38, wherein the method further comprises administration of one or more additional cancer therapies.

40. The method of claim 38 or 39, wherein the subject has or will receive an immunotherapy.

41. The method of any one of claims 38-40, wherein the method further comprises administration of an immunotherapy.

42. The method of claim 40 or 41, wherein the immunotherapy comprises an immune checkpoint inhibitor.

43. The method of claim 42, wherein the immune checkpoint inhibitor comprises an anti-PD-1 monoclonal antibody or an anti-CTLA-4 monoclonal antibody.

44. The method of claim 43, wherein the immune checkpoint inhibitor comprises one or more of nivolumab, pembrolizumab, pidilizumab, ipilimumab or tremelimumab.

45. The method of any one of claims 41-44, wherein the immunotherapy is administered before, after, or concurrent with the polypeptide.

46. The method of any one of claims 38-45, wherein the polypeptide or composition is administered systemically.

47. The method of claim 46, wherein the polypeptide or composition is administered by intravenous injection.

48. The method of any one of claims 38-47, wherein the subject has been previously treated with a cancer therapy.

49. The method of claim 48, wherein the subject has been determined to be non-responsive to the previous treatment or wherein the wherein the subject experienced non-specific toxicity to the previous treatment.
